(19) United States
(12) Patent Application Publication
(10) Pub. No.: US 2013/0236456 A1
(43) Pub. Date: Sep. 12, 2013

(54) IMMUNOGLOBULIN Fc FRAGMENT TAGGING ACTIVATION OF ENDOGENOUS CD4 AND CD8 T CELLS AND ENHANCEMENT OF ANTITUMOR EFFECTS OF LENTIVECTOR IMMUNIZATION

(71) Applicants: Yukai He, Martinez, GA (US); David H. Munn, Augusta, GA (US)

(72) Inventors: Yukai He, Martinez, GA (US); David H. Munn, Augusta, GA (US)

(73) Assignee: Georgia Health Sciences University Research Institute, Inc., Augusta, GA (US)

(21) Appl. No.: 13/788,275

(22) Filed: Mar. 7, 2013

Related U.S. Application Data
(60) Provisional application No. 61/608,359, filed on Mar. 8, 2012.
(60) Provisional application No. 61/618,048, filed on Mar. 30, 2012.
(60) Provisional application No. 61/622,034, filed on Apr. 10, 2012.

(51) Int. Cl. C07K 16/00; C07K 14/02

(52) U.S. Cl. CPC C07K 16/00; C07K 14/02
USPC 424/134.1; 530/387.3; 536/23.4; 435/320.1; 435/69.6

(57) ABSTRACT

A lentivector has been engineered to express a fusion antigen composed of hepatitis B surface protein (HBsAg) and IgG2a Fc fragment (HBS-Fc-Iv) to increase both the magnitude of CD8 response and to induce effective co-activation of CD4 T cells. Immunization with this HBS-Fc-Iv caused significant regression of established tumors. Immunological analysis revealed that, compared to HBS-Iv without the Fc fragment, immunization with HBS-Fc-Iv markedly increased the number of functional CD8 and CD4 T cells and the level of Th1/Tc1-like cytokines in the tumor, while substantially decreasing the Treg ratio. The favorable immunologic changes in tumor lesions and the improvement of antitumor effects from HBS-Fc-Iv immunization were dependent on the CD4 activation, which was Fc receptor mediated. Adoptive transfer of the CD4 T cells from the HBS-Fc-Iv immunized mice could activate endogenous CD8 T cells in an IFNγ-dependent manner. Endogenous CD4 T cells can be activated by lentivirus expressing Fc-tagged antigen to provide another layer of help, i.e., creating a Th1/Tc1 like pro-inflammatory milieu within the tumor lesion to help the effector phase of immune responses to enhance the antitumor effect.

### CROSS-REFERENCE TO RELATED APPLICATIONS

This application claims priority to U.S. Provisional Patent Application Ser. No. 61/608,359 entitled “Fc RECEPTOR TARGETED AND VIRUS-LIKE PARTICLE (VLP) BASED TRIPARTITE VACCINE TECHNOLOGY” and filed Mar. 8, 2012; to U.S. Provisional Patent Application Ser. No. 61/618,048 entitled “Fc RECEPTOR TARGETED AND VIRUS-LIKE PARTICLE (VLP) BASED TRIPARTITE VACCINE TECHNOLOGY” and filed Mar. 30, 2012; and to U.S. Provisional Patent Application Ser. No. 61/622,034 entitled “IMMUNOGLOBULIN Fc FRAGMENT TAGGING ACTIVATION OF ENDOGENOUS CD4 AND CD8 T CELLS AND ENHANCEMENT OF THE ANTITUMOR EFFECT OF LENTIVECTOR IMMUNIZATION” and filed Apr. 10, 2012, the entireties of which are hereby incorporated by reference.

### STATEMENT REGARDING FEDERALLY SPONSORED RESEARCH OR DEVELOPMENT

This invention was made with government support under NIH Grant No. R01 CA116444 awarded by the U.S. National Institutes of Health of the United States government. The government has certain rights in the invention.

### SEQUENCE LISTING

The present disclosure includes a sequence listing incorporated herein by reference in its entirety.

### TECHNICAL FIELD

The present disclosure is generally related to recombinant fusion proteins and to virus-like particles derived therefrom that enhance the activation of CD4 T cells by antigen-presenting cells.

### BACKGROUND

Current tumor immunotherapies heavily rely on the induction of CD8 immune responses. Peptide—(Elsawa et al., (2004) Expert Rev. Vaccines 3: 563-5751), dendritic cell—(Melief, C. J. (2008) Immunity 29: 372-383), and viral vector—(Anderson & Schneider (2007) Vaccine 25: 624-34; Harrop et al. (2006) Adv. Drug Delivery Revs. 58: 931-947) based cancer vaccines have been demonstrated to activate and expand tumor-specific CD8 T cells. It has been found (He & Falo, Jr. (2007) Curr. Opin. Mol. Ther. 9: 439-446; He et al., (2006) Immunity 24: 643-656; He et al., (2005) J. Immunol. 174: 3808-3817; Zhou et al., (2010) J. Immunol. 185: 5082-5092; Liu et al., (2009) J. Immunol. 182: 5960-5969; Dullaers et al. (2006) Gene Ther. 13: 630-640; Esslinger et al., (2003) J. Clin. Invest. 111: 1673-1681; Rowe et al., (2006) Mol. Ther. 13: 310-319) that recombinant lentivector can effectively stimulate CD8 responses due to its efficient transduction of migrating skin dendritic cells that are able to directly prime naïve CD8 T cells in the draining lymph nodes (He et al., (2006) Immunity 24: 643-656; Furmanov et al., (2010) J. Immunol. 184: 4889-4897). However, despite these intensive efforts and the induction of potent CD8 responses, the antitumor effect of current cancer vaccines in treating established tumors is limited (Liu et al. (2009) J. Immunol. 182: 5960-5969; Rosenberg et al., (2004) Nature Medicine 10: 909-915; Singh et al., (2009) J. Immunother. 32: 129-139). In contrast to CD8 T cells, the activation of CD4 T cells is more difficult (Seder & Ahmed (2003) Nat. Immunol. 4: 835-842), especially when the production of effector cytokines by CD4 T cells is used as a criterion of activation.

As a helper T cell, CD4 is widely recognized for its role in helping the induction of CD8 responses by possibly “licensing” dendritic cells (Castellino & Germain (2006) Ann. Rev. Immunol. 24: 519-540; Ridge et al., (1998) Nature 393: 474-478; Schoenberger et al., (1998) Nature 393: 480-483; Bennett et al., (1998) Nature 393: 478-480; Lanzavecchia, A. (1998) Nature 393: 413-414). In addition, the “post-licensing” role of CD4 T cells at the effector phase is becoming increasingly appreciated (Kennedy & Celis (2008) Immunol. Rev. 222: 129-144). Recent studies showed that the recruitment, proliferation, and effector functions of CD8 T cells inside tumors or infected lesions could be greatly enhanced by co-transfer of CD4 T cells (Bos & Sherman (2010) Cancer Res. 70: 8368-8377; Marzo et al., (2000) J. Immunol. 165: 6047-6055; Nakanishi et al., (2009) Nature 462: 510-513). Furthermore, several reports have shown that some CD4 T cells could directly kill tumor cells (Xie et al., (2010) J. Exp. Med. 207: 651-667; Quezada et al., (2010) J. Exp. Med. 207: 637-650; Ding et al., (2010) Blood 115: 2397-2406; Perez-Diez et al., (2007) Blood 109: 5346-5354). However, in most, if not all, of these studies, adoptive transfer of a large number of highly selected T cell receptor (TCR) transgenic (Tg) CD4 T cells were utilized.

It is not clear if the rare endogenous CD4 T cells can exert similar functions because most of the current cancer vaccines, especially those that are gene-based, are not effective at activating CD4 T cells. Although proven to be very effective at stimulating CD8 responses, lentivirus (Iv) immunization has limited ability to activate endogenous CD4 T cells. It has been demonstrated that CD4 T cells could be activated by lentivirus immunization only by adoptive transfer of a high number of exogenous TCR Tg OT-II CD4 T cells (Rowe et al., (2006) Mol. Ther. 13: 310-319). In another study, it was demonstrated that a limited activation of endogenous OVA-specific CD4 T cells was achieved after lentivirus immunization when OVA antigen was channeled to MHC II restricted pathway (Goold et al. (2011) J. Immunol. 186: 4565-4572).

### SUMMARY

A major problem with current cancer vaccines is that the induction of CD8 immune responses is rarely associated with antitumor benefits mainly due to the multiple immune suppressions in the established tumor lesions. A lentivector has now been engineered to express a fusion antigen composed of hepatitis B surface protein (HBsAg) and IgG2a Fc fragment (HBS-Fc-Iv) to increase both the magnitude of CD8 response and to induce effective co-activation of CD4 T cells. Immunization with this HBS-Fc-Iv caused significant regression of established tumors. Immunological analysis revealed that, compared to HBS-Iv without the Fc fragment, immunization with HBS-Fc-Iv markedly increased the number of functional CD8 and CD4 T cells and the level of Th1/Tc1-like cytokines in the tumor, while substantially decreasing the Treg ratio. The favorable immunologic changes in tumor lesions and the improvement of antitumor effects from HBS-Fc-Iv immunization were dependent on the CD4 activation, which was Fc receptor mediated. Adoptive transfer of the CD4 T cells from the HBS-Fc-Iv immunized mice could activate endogenous CD8 T cells in an IFNγ-dependent manner. Endogenous CD4 T cells can be activated by lentivirus expressing Fc-tagged antigen to provide another layer of help, i.e., creating a Th1/Tc1 like pro-inflammatory milieu within the tumor lesion to help the effector phase of immune responses to enhance the antitumor effect.

One aspect of the present disclosure, therefore, encompasses embodiments of a recombinant fusion protein for potentiating an immune system, the fusion protein comprising a self-assembling virus-like particle-forming polypeptide; an Fcγ receptor (FcγR)-binding ligand polypeptide, and a target antigen polypeptide, thereby forming a tripartite fusion polypeptide. In the embodiments of this aspect of the disclosure, the target antigen polypeptide is positioned between the self-assembling virus-like particle-forming polypeptide and the Fcγ receptor (FcγR)-binding ligand polypeptide.

In the embodiments of this aspect of the disclosure, the self-assembling virus-like particle-forming polypeptide is selected from the group consisting of: an HPV L1 protein, an HPV L2 protein, an influenza Hemagglutinin A, an influenza neuraminidase, an influenza M1 protein, a lentivirus protein, any self-assembling fragment thereof, or any combination thereof.

In the embodiments of this aspect of the disclosure, the target antigen polypeptide is a hepatitis B surface antigen (HBsAg) polypeptide, or fragment thereof.

In the embodiments of this aspect of the disclosure, the immunoglobulin Fcγ receptor-binding ligand domain can comprise an immunoglobulin Fc domain.

In the embodiments of this aspect of the disclosure, the immunoglobulin Fcγ receptor-binding ligand domain can be isolated from an immunoglobulin G2a (IgG2a) or an immunoglobulin G1 (IgG1).

Another aspect of the disclosure encompasses embodiments of a nucleic acid encoding a recombinant fusion polypeptide as disclosed herein.

In the embodiments of this aspect of the disclosure, the nucleic acid can be inserted into an expression vector, and wherein the nucleic acid is operably linked to an expression control region for expression of the nucleic acid in a recipient cell.

In the embodiments of this aspect of the disclosure, the nucleic acid can be within a host cell selected from a mammalian cell, an insect cell, a yeast cell, and a prokaryotic cell. Another aspect of the disclosure encompasses embodiments of an immunopotentiating virus-like particle (VLP) comprising a recombinant fusion protein according to the present disclosure.

Still another aspect of the disclosure encompasses embodiments of a method of generating a plurality of virus-like particles (VLP) comprising a recombinant fusion protein, the method comprising: (a) providing a nucleic acid encoding a recombinant fusion protein according to the disclosure, where the nucleic acid is inserted into an expression vector, and where the nucleic acid is operably linked to an expression control region for expression of the nucleic acid in a recipient cell; (b) delivering the nucleic acid to a host cell suitable for expression of the nucleic acid from the expression vector, where the host cell is selected from a mammalian cell, an insect cell, a yeast cell, and a prokaryotic cell; (c) expressing the nucleic acid in the host cell to provide said recombinant fusion protein; and (d) allowing the recombinant fusion protein to self-assemble to form a plurality of VLPs.

In the embodiments of this aspect of the disclosure, the method can further comprise the step of combining the plurality of VLPs with a physiologically acceptable carrier.

In the embodiments of this aspect of the disclosure, the method can further comprise the step of combining the plurality of VLPs with an adjuvant.

Still another aspect of the disclosure encompasses embodiments of an immunogenic composition comprising a plurality of immunopotentiating VLPs, wherein the immunopotentiating VLPs comprise a recombinant fusion protein of the present disclosure, wherein said immunogenic composition is devoid of an adjuvant.

Another aspect of the disclosure encompasses embodiments of an immunogenic composition comprising a plurality of immunopotentiating VLPs, where the immunopotentiating VLPs can comprise a recombinant fusion protein according to the disclosure, where the immunogenic composition can further comprise at least one adjuvant.

Another aspect of the disclosure encompasses embodiments of a method of inducing an immune response in an animal or human subject, comprising administering to the animal or human subject an immunogenic composition comprising a plurality of immunopotentiating VLPs comprising a recombinant fusion protein according to the present disclosure, whereby the VLPs are ingested by an antigen-presenting cell, thereby activating CD4 and CD8 T cells by MHC I and II.

In the embodiments of this aspect of the disclosure, the method can further comprise the step of allowing the activated CD4 and CD8 T cells to invade a tumor, thereby reducing the extent of the tumor in the recipient animal or human subject.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIG. 1 illustrates that lentivirus (Iv) expressing Fc-tagged antigen elicits more potent CD8 and CD4 T cell immune responses. C57BL/6 mice were immunized with either HBS-Iv or HBS-Fc-Iv. Non-immunized mice were used as control. Two weeks later, HBsAg-specific CD8 and CD4 T cell responses in the peripheral blood were determined by intracellular staining of IFNγ after brief stimulation ex vivo with S₁₉₀₋₁₉₇ peptide (for CD8 response) or whole HBsAg (for CD4 responses). Only CD8 or CD4 T cells were gated (top). Data from 5 mice in each group were summarized and shown in the graphs (bottom). The experiment was repeated three times with similar results.

FIGS. 2A and 2B illustrate that HBS-Fc-Iv immunization results in regression of established B16-S tumors. FIG. 2A shows the experimental design of tumor treatment with lentivirus immunization. FIG. 2B shows the growth curve of lentivirus-treated and control tumors.

FIGS. 3A and 3B illustrate that tumor lesion is skewed toward a Th1/Tc1-like immune stimulatory microenvironment after HBS-Fc-Iv immunization. Eighteen days after immunization, total RNA was extracted from the tumor tissues of control and immunized mice (3 tumor lesions in each group were combined together), and qRT-PCR was performed using primers for indicated cytokines, transcription factors, and chemokines. The results are presented as folds of increase of cytokines (FIG. 3A) or chemokines (FIG. 3B) in the immunized tumor over the control tumors. Each sample was done in triplicate; the average and SD are shown. The experiment was repeated 3 times with similar results.

FIGS. 4A-4C illustrate that HBS-Fc-Iv immunization markedly increases tumor infiltration of CD4 and CD8 T cells and decreases the Treg ratio in the tumor lesions. Mice bearing 5-day tumors were immunized with either HBS-Iv or HBS-Fc-Iv, or left untreated. The tumor lesions were collected on day 17-20 after immunization and analyzed for tumor-infiltrating CD8 (FIG. 4A) and CD4 (FIG. 4B) T cells and the Treg ratio (FIG. 4C). The absolute numbers of CD8 and CD4 T cells and the Treg ratios in the tumor lesions of control and treated mice from a cohort of two studies are summarized.

FIGS. 5A-5C illustrate that CD4 and CD8 TIL in the tumors treated with HBS-Fc-Iv possess better effector function. FIG. 5A illustrates the effector function of CD8 TIL measured by intracellular staining of IFNγ after brief ex vivo peptide stimulation. Representative dot plots of CD8 TIL from HBS-Iv- or HBS-Fc-Iv-immunized tumors are shown. Only the CD8 T cells were gated and shown. Data of 5 mice are summarized. FIG. 5B illustrates effector cytokine production by CD4 TILs after stimulation with PMA/Ionomycin. Only the CD4 T cells were gated and shown. These experiments examining the TIL and their effector functions were repeated 3 times with similar results. FIG. 5C illustrates the degranulation of CD8 TIL measured by CD107a staining. A summary of data from 5 tumors in each group is presented. This experiment was repeated twice with similar observations.

FIGS. 6A-6D illustrate that immunologic changes in the tumors and the antitumor effect of HBS-Fc-Iv immunization are dependent on CD4 activation and FcγR. Wild-type and FcRγ knock-out mice were inoculated with B16-S tumor cells. Five days later, tumor-bearing mice were immunized with HBS-Fc-Iv. FIG. 6A illustrates CD8 and CD4 responses in the peripheral blood determined 2 weeks after immunization by intracellular staining of IFN₁. Data of 5 mice are shown on the right. FIG. 6B illustrates the Treg ratio in the tumor analyzed 3 weeks after tumor inoculation and the data of 5 mice are summarized at the right column. FIG. 6C illustrates cytokines in the tumor lesions of wild-type and knockout mice analyzed by qRT-PCR. FIG. 6D illustrates the tumor growth curve and the tumor weight. Two experiments were conducted with similar results.

FIGS. 7A-7D illustrate that adoptive transfer of CD4 T cells can activate endogenous CD8 TIL in tumors and generate antitumor effects. FIG. 7A illustrates the experimental scheme: Activated CD4 and CD8 T cell subsets were isolated from HBS-Fc-Iv-immunized Thy1.1 congenic mice and 10 million cells were adoptively transferred into mice (bearing 5-day B16-S tumors) after a low dose (5Gy) irradiation. Two weeks after adoptive transfer, the tumor lesions were collected and the granzyme B (GrzB) expression of TILs was analyzed. FIG. 7B illustrates GrB expression of endogenous CD8 TIL from mice transferred with activated CD4 or CD8 T cells. Irradiated mice without adoptive transfer of T cells were used as control. A summary of data of total CD8 TIL and percentage of GrzB expression is also shown. FIG. 7C illustrates GrzB expression of exogenous adopted CD4 and CD8 TILs. A summary of data is shown on the right. FIG. 7D shows tumor growth curve and tumor weight. A summary of data from 5 mice is shown. Two experiments were conducted with similar results.

FIGS. 8A-8D illustrate that IFNγ expression plays a critical role in the antitumor effect of CD4 T cell adoptive transfer. FIG. 8A shows the experimental design. Wild-type and IFNγ knockout mice (Thy1.2) were immunized with HBS-Fc-Iv. Two weeks later, activated CD4 T cells were isolated and 5 million cells were injected into irradiated B16-S tumor-bearing mice (Thy1.1). Mice with irradiation only without cell transfer were used as control. FIG. 8B shows activation of CD4 T cells by HBS-Fc-Iv immunization in both wild-type and IFNγ knockout mice as determined by examining TNFα expression. A similar percent of CD4 T cells of wild-type and IFNγ knockout mice produced TNFα in responses to antigen stimulation. FIG. 8C shows the absolute number of IFNγ-producing CD8 TIL of 5 mice in two experiments two weeks after transfer. FIG. 8D shows the tumor weights recorded when the mice were sacrificed at the end of the experiment.

FIG. 9 illustrates the amino acid sequence (SEQ ID No.: 1) of the HBS-Fc fusion antigen. The Fc fragment (CH₂₋₃ domain) of mouse IgG2a is underlined.

FIGS. 10A and 10B illustrate the infiltration of CD8 and CD4 TIL is markedly increased by immunization with HBS-Fc-Iv. FIG. 10A shows the gating strategy. FIG. 10B shows representative dot plots of CD4 and CD8 TIL and Treg ratio from the tumors of different treatment groups.

FIGS. 11A and 11B illustrate the adoptive transfer of CD4 T cells from HBS-Fc-Iv-immunized mice induces more tumor infiltration of endogenous CD8 T cells and stronger CD8 TIL activation, resulting in enhanced anti-tumor effect. FIG. 11A shows CD4 T cells isolated from mice immunized with HBS-Fc-IV, HBS-Iv, or naïve mice, and 5 million cells were transferred into irradiated (5Gy) tumor-bearing mice. Tumor growth was monitored and tumor weight was recorded when the mice were sacrificed. FIG. 11B shows the effector function of endogenous CD8 TIL from tumor-bearing mice transferred with above CD4 T cells analyzed for granzyme B expression and the absolute number of CD8 TIL enumerated. Only the endogenous CD8 TILs were gated and shown in the dot plot. Two experiments were repeated with similar data.

FIGS. 12A and 12B illustrate tagging of HBsAg with the Fc fragment of mouse IgG2a increases the HBsAg level. FIG. 12A shows a schematic structure of recombinant lentivector HBS-Iv and HBS-Fc-Iv. The HBsAg and HBS-Fc fusion construct was inserted behind the CMV promoter in the recombinant lentivector. FIG. 12B shows the increase of expression and secretion of HBsAg after transduction. 293T cells were transduced with either HBS-Iv or HBS-Fc-Iv lentivector. Untransduced 293T cells were used as control. Two days after transduction, HBsAg levels in the supernatant and cell lysate were examined by ELISA. The data value was presented as OD₄₅₀ in the total cell lysate or supernatant. The mean±SEM is indicated on the figure by the central value and error bar (95% confidence limit). Statistical analysis was done using unpaired t-test. The experiment was repeated three times with similar data.

FIGS. 13A and 13B illustrate lentivector HBS-Fc-Iv immunization stimulates potent CD8 T cell responses. Female C57BL/6 mice (5 mice in each group) were immunized with lentivector HBS-Iv or HBS-Fc-Iv on footpad. For DNA immunization, intramuscular injection was performed. Two weeks later, immune responses were examined. FIG. 13A shows intracellular staining of the IFNγ among CD8 T cells in the peripheral blood after brief (3 h) ex vivo stimulation with HBS peptide. The numbers on the upper right quadrant represent the percentage of IFNγ-producing CD8 T cells out of total CD8 T cells. FIG. 13B shows the in vivo CTL activity assayed on day 14 by an in vivo killing assay. Only the CFSE+ cells were shown in the histogram. The numbers represent the percentage of each CFSE+ cell population. The experiment was reproduced at least 5 times with similar observation. FIG. 13C shows a summary of the IFNγ+CD8 from 5 mice in each group. The mean±SEM is indicated by the central value and error bar (95% confidence limit). Unpaired t-test was used for statistical analysis.

FIGS. 14A and 14B illustrate lentivector HBS-Fc-Iv immunization elicits CD4 T cell responses and humoral immune responses. FIG. 14A shows CD4 T cell responses when peripheral blood cells were stimulated with HBsAg whole protein before intracellular staining of IFNγ and surface staining of CD4. Only the Thy1.2⁺ CD4⁺ T cells are shown. The numbers indicate the percentage of each cell population. A summarized data from 5 mice is also shown. FIG. 14B shows the humoral immune response determined by the anti-HBsAb level using ELISA. The data are presented as OD₄₅₀/ml of serum. A summary of 10 immunized mice is presented. The mean±SEM is indicated on the figure by the central value and error bar (95% confidence limit). Unpaired t-test was used for analysis.

FIGS. 15A-15C illustrate that the CD4 and humoral immune responses but not CD8 T cell responses are affected by the Fc receptor. To determine if the HBS-Fc-Iv-induced immune responses were mediated by Fc receptor, five wild-type C57BL/6 mice and FcRγ knockout (also on C57BL/6 background) mice were immunized with HBS-Fc-Iv. FIG. 15A shows the CD8 T cell response was not affected by Fc receptor knockout. FIGS. 15B and 15C show that the CD4 T cell response and humoral immune response were severely compromised in the Fc receptor knockout mice. Only the indicated cell population is shown in the dot plot (FIG. 15B, left). The anti-HBs Ab value is presented as OD₄₅₀/ml of serum. A summary of data from 5 mice is presented. The mean±SEM is indicated on the figure by the central value and error bar (95% confidence limit). Unpaired t-test was used for statistical analysis.

FIGS. 16A-16C illustrate HBS-Fc-Iv immunization can break immune tolerance in Tg mice expressing a low level of HBsAg. FIG. 16A shows offspring of HBsAg Tg mice from Jackson Laboratory could be divided into two groups based on the serum level of HBsAg (FIG. 16A, upper): HBsAg^{high} (OD₄₅₀>1.0) and HBsAg^{low} (0.1>OD₄₅₀>0.05) mice; the cutoff value of OD₄₅₀ is 0.0405. The HBsAg level in the liver of HBsAg Tg mice was also determined. Although significantly lower than HBsAg^{high}, Tg-lo mouse liver contained a definitive level of HBsAg (FIG. 16A, bottom). FIG. 16B shows that following lentivector HBS-Fc-Iv immunization, CD8 T cell immune responses were detected in peripheral blood and liver of both wild-type (normal C57BL/6) mice and HBsAg^{low} Tg mice. No CD8 response was detected in the HBsAg^{high} Tg mice. A summary of 5 mice is also presented. Unpaired t-test was utilized for analysis. FIG. 16C shows in vivo CTL activity determined by in vivo killing assay. The Ctrl mice (normal C57BL/6 without treatment) and the HBsAg^{high} Tg mice did not show any in vivo killing activity, while immunized wild-type mice had a high level of CTL activity. A lower level of in vivo killing activity was detected in the HBsAg^{low} Tg mice. A summary of 4 mice is also shown. The mean±SEM is indicated on the figure by the central value and error bar (95% confidence limit). Unpaired t-test was used for analysis.

FIGS. 17A-17C illustrate HBS-Fc-Iv immunization results in seroconversion of HBsAg to anti-HBsAb. FIG. 17A shows HBS-Fc-Iv immunization reduced the serum HBsAg in the HBsAg^{low} Tg mice to become negative (left column). FIG. 17B shows that anti-HBs Ab could be detected in the HBsAg^{low} Tg mouse serum only after HBS-Fc-Iv immunization. Data from 7 mice is presented. The ELISA data value is presented as OD₄₅₀/ml of serum. FIG. 17C shows that compared to control (non-immunized) HBsAg^{low} Tg mice, no significant increase of serum ALT was found in the immunized HBsAg^{low} Tg mice. The mean±SEM is indicated on the figure by the central value and error bar (95% confidence limit). Statistical analysis was performed using unpaired t-test.

FIG. 18A illustrates the HBS-TRP1-Fc Tripartite Ag structure.

FIG. 18B shows the amino acid sequence (SEQ ID No.: 2) of the HBsAg-TRP-1-Fc fusion antigen.

FIG. 19 illustrates the nucleotide sequence (SEQ ID No.: 3) encoding the HBsAg-TRP-1-Fc fusion antigen.

FIG. 20 illustrates that TRP1 peptides in the form of the HBS-TRP1-Fc tripartite fusion protein stimulated the most potent TRP1-specific CD8 and CD4 response. The recombinant lentivirus expressed the 65aa of TRP1 only, Fc-tagged TRP1 (TRP1-Fc), HBsAg-fused TRP1 (HBS-TRP1), or the HBS-TRP1-Fc tripartite Ag. A representative dot plot of CD8 response (IFNγ production) from each lentivirus immunization is shown (top). A summary of 5 mice is presented at the left. Only the CD8 T cells were gated and shown. To detect CD4 responses, TRP1-specific CD4 TCR Tg T cells (Thy1.2+) were adoptively transferred into Thy1.1 B6 mice. The Thy1.2+ CD4 T cells were examined 8 days after immunization (bottom).

FIG. 21 illustrates a schematic diagram of the structures of recombinant lentivectors expressing the HBsAg-AFP-Fc tripartite fusion Ag. A: Two epitope-optimized mouse alpha fetoprotein (mAFP) fragments of mAFP142 (Amino acid residues 152-293) and mAFP164 (Amino acid residues 411-574) were cloned into recombinant lentivector to generate mAFP₁₄2-Iv and mAFP₁₆₄-Iv, respectively. B: The full length HBsAg (capable of forming VLP) were fused to the N-terminus of mAFP142 or mAFP164, which were then cloned into lentivector to generate S-A142-Iv and S-A164-Iv. C: The S-A142 and S-A164 bipartite Ag were further fused to the Fc fragment of immunoglobulin G at the C-terminus to create recombinant lentivector S-A142-Fc-Iv and S-AFP164-Fc-Iv, respectively.

FIG. 22 illustrates recombinant lentivector expressing HBsAg-mAFP-Fc tripartite fusion antigen stimulates the most potent mAFP-specific immune responses. B6 mice were immunized with recombinant lentivector expressing mAFP fragments, HBsAg-mAFP bipartite, or HBsAg-mAFP-Fc tripartite fusion antigen. After two weeks, the mAFP-specific CD8 immune responses of circulating blood cells were examined using intracellular staining of IFNγ after brief 4 h ex vivo stimulation with mAFP epitope AFP₂₁₂ (upper) or AFP₄₉₉ (lower). Immunization with recombinant lentivector expressing HBsAg-mAFP-Fc induced the most potent mAFP-specific CD8 responses.

FIGS. 23A and 23B illustrate the nucleotide (FIG. 23A) (SEQ ID NO.: 4) and amino acid (FIG. 23B) (SEQ ID NO.: 5) sequences of HBsAg-mAFP-Fc (S-A142-Fc) tripartite fusion Ag. The underlined regions indicate the optimized mAFP fragment (152-293aa). Sequences in front of the mAFP are the HBsAg. Sequences after the mAFP are the Fc fragment of immunoglobulin G2a (mlgG2a).

FIGS. 24A and 24B illustrate the nucleotide (FIG. 24A) (SEQ ID No. 6) and amino acid (SEQ ID NO.: 7) (FIG. 24B) sequences of HBsAg-mAFP-Fc (S-A164-Fc) tripartite fusion Ag. The underlined region indicates the optimized mAFP fragment (411-574aa). Sequences in front of the mAFP are the HBsAg. Sequences after the mAFP are the Fc fragment of immunoglobulin G2a (mlgG2a).

The drawings are described in greater detail in the description and examples below.

The details of some exemplary embodiments of the methods and systems of the present disclosure are set forth in the description below. Other features, objects, and advantages of the disclosure will be apparent to one of skill in the art upon examination of the following description, drawings, examples and claims. It is intended that all such additional systems, methods, features, and advantages be included within this description, be within the scope of the present disclosure, and be protected by the accompanying claims.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

As used herein, the following terms have the meanings ascribed to them unless specified otherwise. In this disclosure, “comprises,” “comprising,” “containing” and “having” and the like can have the meaning ascribed to them in U.S. patent law and can mean “includes,” “including,” and the like; “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein.

### ABBREVIATIONS

Iv: lentivector; HBsAg: Hepatitis B virus surface Ag; HBS-Fc: HBsAg and mouse IgG2a Fc fragment fusion Ag; HBS-Fc-Iv: lentivector expressing HBS-Fc fusion Ag; FcγR: Fcγ receptor; FcRγ: Fc receptor γ chain; KO: knockout; WT: wild type; ACT: adoptive cell transfer.

### DEFINITIONS

In describing and claiming the disclosed subject matter, the following terminology will be used in accordance with the definitions set forth below.

Unless otherwise defined, all terms of art, notations and other scientific terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art, such as, for example, the widely utilized molecular cloning methodologies described in Sambrook et al. Molecular Cloning: A Laboratory Manual 3rd. edition (2001) Cold Spring Harbor Laboratory Press, Cold Spring Harbor, N.Y. and Current Protocols in Molecular Biology (Ausbel et al., eds., John Wiley & Sons, Inc. 2001). As appropriate, procedures involving the use of commercially available kits and reagents are generally carried out in accordance with manufacturer defined protocols and/or parameters unless otherwise noted.

The term “adjuvant molecule” as used herein refers to surface proteins capable of eliciting an immune response in a host. In particular embodiments, the adjuvant molecule is a “membrane-anchored form” of the adjuvant molecule which indicates that the adjuvant molecule has been engineered to include a signal peptide (SP) and a membrane anchor sequence to direct the transport and membrane orientation of the protein. Thus, in embodiments, a membrane-anchored form of an adjuvant molecule is a recombinant protein including a portion of a protein fused to a SP and membrane anchor sequence.

The term “administration” as used herein refers to introducing a composition (e.g., a vaccine, adjuvant, or immunogenic composition) of the present disclosure into a subject. The preferred route of administration of the vaccine composition is intravenous. However, any route of administration, such as oral, topical, subcutaneous, peritoneal, intra-arterial, inhalation, vaginal, rectal, nasal, introduction into the cerebrospinal fluid, or instillation into body compartments, can be used.

The term “antigen” as used herein refers to a molecule containing one or more epitopes (either linear, conformational or both) that will stimulate a host's immune system to make a humoral and/or cellular antigen-specific response. The term is used interchangeably with the term “immunogen.” Normally, a B-cell epitope will include at least about 5 amino acids but can be as small as 3-4 amino acids. A T-cell epitope, such as a CTL epitope, will include at least about 7-9 amino acids, and a helper T-cell epitope at least about 12-20 amino acids. Normally, an epitope will include between about 7 and 15 amino acids, such as, 9, 10, 12 or 15 amino acids. The term includes polypeptides which include modifications, such as deletions, additions and substitutions (generally conservative in nature) as compared to a native sequence, so long as the protein maintains the ability to elicit an immunological response, as defined herein. These modifications may be deliberate, as through site-directed mutagenesis, or may be accidental, such as through mutations of hosts which produce the antigens.

The term “cancer,” as used herein shall be given its ordinary meaning and is a general term for diseases in which abnormal cells divide without control. Cancer cells can invade nearby tissues and can spread through the bloodstream and lymphatic system to other parts of the body. There are several main types of cancer, for example, carcinoma is cancer that begins in the skin or in tissues that line or cover internal organs. Sarcoma is cancer that begins in bone, cartilage, fat, muscle, blood vessels, or other connective or supportive tissue. Leukemia is cancer that starts in blood-forming tissue, such as the bone marrow, and causes large numbers of abnormal blood cells to be produced and enter the bloodstream. Lymphoma is cancer that begins in the cells of the immune system.

When normal cells lose their ability to behave as a specified, controlled and coordinated unit, a tumor is formed. Generally, a solid tumor is an abnormal mass of tissue that usually does not contain cysts or liquid areas (some brain tumors do have cysts and central necrotic areas filled with liquid). A single tumor may even have different populations of cells within it with differing processes that have gone awry. Solid tumors may be benign (not cancerous) or malignant (cancerous). Different types of solid tumors are named for the type of cells that form them. Examples of solid tumors are sarcomas, carcinomas, and lymphomas. Leukemias (cancers of the blood) generally do not form solid tumors. Representative cancers include, but are not limited to, bladder cancer, breast cancer, colorectal cancer, endometrial cancer, head and neck cancer, leukemia, lung cancer, lymphoma, melanoma, non-small-cell lung cancer, ovarian cancer, prostate cancer, testicular cancer, uterine cancer, and cervical cancer.

The term “chimeric” viral surface proteins are ones that contain at least a portion of the extracellular domain of a viral surface protein of a virus and at least a portion of domains and/or signal peptide sequence of a different protein. Such chimeric proteins retain surface antigenic determinants against which an immune response is generated, preferably a protective immune response, and retain sufficient envelope sequence for proper precursor processing and membrane insertion. The skilled artisan can produce chimeric viral surface proteins using recombinant DNA technology and protein coding sequences, techniques known to those of skill in the art and available to the public.

The term “coding sequence” or a sequence which “encodes” a selected polypeptide as used herein refers to a nucleic acid molecule which is transcribed (in the case of DNA) and translated (in the case of mRNA) into a polypeptide in vivo when placed under the control of appropriate regulatory sequences (or “control elements”). The boundaries of the coding sequence are determined by a start codon at the 5′ (amino) terminus and a translation stop codon at the 3′ (carboxy) terminus. A coding sequence can include, but is not limited to, cDNA from viral, prokaryotic or eukaryotic mRNA, genomic DNA sequences from viral or prokaryotic DNA, and even synthetic DNA sequences. A transcription termination sequence may be located 3′ to the coding sequence.

The term “control elements” as used herein refers to, but is not limited to, transcription promoters, transcription enhancer elements, transcription termination signals, polyadenylation sequences (located 3′ to the translation stop codon), sequences for optimization of initiation of translation (located 5′ to the coding sequence), and translation termination sequences, see, e.g., McCaughan et al., (1995) Proc. Natl. Acad. Sci. U.S.A. 92: 5431-5435; Kochetov et al., (1998) FEBS Letts. 440: 351-355.

The term “derived from” as used herein refers to the origin or source, and may include naturally occurring, recombinant, unpurified, or purified molecules. The proteins and molecules of the present disclosure may be derived from, but not limited to, a lentivirus or lentivirus vector.

The term “DNA” as used herein refers to the polymeric form of deoxyribonucleotides (adenine, guanine, thymine, or cytosine) in either single-stranded form or as a double-stranded helix. This term refers only to the primary and secondary structure of the molecule and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA).

The term “DNA amplification” as used herein refers to any process that increases the number of copies of a specific DNA sequence by enzymatically amplifying the nucleic acid sequence. A variety of processes are known. One of the most commonly used is the polymerase chain reaction (PCR), which is defined and described in later sections below. The PCR process of Mullis is described in U.S. Pat. Nos. 4,683,195 and 4,683,202. PCR involves the use of a thermostable DNA polymerase, known sequences as primers, and heating cycles, which separate the replicating deoxyribonucleic acid (DNA) strands and exponentially amplify a gene of interest. Any type of PCR, such as quantitative PCR, RT-PCR, hot start PCR, LAPCR, multiplex PCR, touchdown PCR, etc., may be used. Advantageously, real-time PCR is used. In general, the PCR amplification process involves an enzymatic chain reaction for preparing exponential quantities of a specific nucleic acid sequence. It requires a small amount of a sequence to initiate the chain reaction and oligonucleotide primers that will hybridize to the sequence. In PCR the primers are annealed to denatured nucleic acid followed by extension with an inducing agent (enzyme) and nucleotides. This results in newly synthesized extension products. Since these newly synthesized sequences become templates for the primers, repeated cycles of denaturing, primer annealing, and extension results in exponential accumulation of the specific sequence being amplified. The extension product of the chain reaction will be a discrete nucleic acid duplex with a termini corresponding to the ends of the specific primers employed.

The term “epitope” as used herein refers to the site on an antigen that is recognized by a T-cell receptor and/or an antibody.

The terms “expressed” and “expression” as used herein refer to the transcription from a gene to give an RNA nucleic acid molecule at least complementary in part to a region of one of the two nucleic acid strands of the gene. The term “expressed” or “expression” as used herein also refers to the translation from said RNA nucleic acid molecule to give a protein, an amino acid sequence or a portion thereof.

The term “expression vector” as used herein refers to a nucleic acid useful for expressing the DNA encoding the protein used herein and for producing the protein. The expression vector is not limited as long as it expresses the gene encoding the protein in various prokaryotic and/or eukaryotic host cells and produces this protein. When yeast, animal cells, or insect cells are used as hosts, an expression vector preferably comprises, at least, a promoter, an initiation codon, the DNA encoding the protein and a termination codon. It may also comprise the DNA encoding a signal peptide, enhancer sequence, 5′- and 3′-untranslated region of the gene encoding the protein, splicing junctions, polyadenylation site, selectable marker region, and replicon. The expression vector may also contain, if required, a gene for gene amplification (marker) that is usually used.

A promoter/operator region to express the protein in bacteria comprises a promoter, an operator, and a Shine-Dalgarno (SD) sequence (for example, AAGG). For example, when the host is Escherichia, it preferably comprises Trp promoter, lac promoter, recA promoter, lambda.PL promoter, b 1pp promoter, tac promoter, or the like. When the host is a eukaryotic cell such as a mammalian cell, examples thereof are SV40-derived promoter, retrovirus promoter, heat shock promoter, and so on. As a matter of course, the promoter is not limited to the above examples. In addition, using an enhancer is effective for expression. A preferable initiation codon is, for example, a methionine codon (ATG). A commonly used termination codon (for example, TAG, TAA, and TGA) is exemplified as a termination codon. Usually, used natural or synthetic terminators are used as a terminator region. An enhancer sequence, polyadenylation site, and splicing junction that are usually used in the art, such as those derived from SV40, can also be used. A selectable marker usually employed can be used according to the usual method. Examples thereof are resistance genes for antibiotics, such as tetracycline, ampicillin, or kanamycin.

The expression vector used herein can be prepared by continuously and circularly linking at least the above-mentioned promoter, initiation codon, DNA encoding the protein, termination codon, and terminator region to an appropriate replicon. If desired, appropriate DNA fragments (for example, linkers, restriction sites, and so on) can be used by a method such as digestion with a restriction enzyme or ligation with T4 DNA ligase. Transformants can be prepared by introducing the expression vector mentioned above into host cells.

The term “fragment” of a molecule such as a protein or nucleic acid as used herein refers to any portion of the amino acid or nucleotide genetic sequence.

The term “host” or “subject” as used herein refers to humans, other mammals (e.g., cats, dogs, horses, etc.), living cells, and other living organisms. A living organism can be as simple as, for example, a single eukaryotic cell or as complex as a mammal. Typical hosts to which embodiments of the present disclosure may be administered will be mammals, particularly primates, and especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals, particularly pets such as dogs and cats. For diagnostic or research applications, a wide variety of mammals will be suitable subjects, including rodents (e.g., mice, rats, hamsters), rabbits, primates, and swine such as inbred pigs and the like.

The term “identity” as used herein refers to a relationship between two or more polypeptide sequences, as determined by comparing the sequences. In the art, “identity” also refers to the degree of sequence relatedness between polypeptides as determined by the match between strings of such sequences. “Identity” and “similarity” can be readily calculated by known methods, including, but not limited to, those described in Computational Molecular Biology, Lesk, A. M., Ed., Oxford University Press, NY, 1988; Biocomputing: Informatics and Genome Projects, Smith, D. W., Ed., Academic Press, NY, 1993; Computer Analysis of Sequence Data, Part I, Griffin, A. M. & and Griffin, H. G., Eds., Humana Press, NJ, 1994; Sequence Analysis in Molecular Biology, von Heinje, G., Academic Press, 1987; Sequence Analysis Primer, Gribskov, M. & Devereux, J., Eds., M Stockton Press, NY, 1991; and Carillo & Lipman (1988) SIAM J. Applied Math., 48: 1073.

Preferred methods to determine identity are designed to give the largest match between the sequences tested. Methods to determine identity and similarity are codified in publicly available computer programs. The percent identity between two sequences can be determined by using analysis software (i.e., Sequence Analysis Software Package of the Genetics Computer Group, Madison, Wis.) that incorporates the Needelman & Wunsch ((1970) J. Mol. Biol., 48: 443-453) algorithm (e.g., NBLAST and XBLAST). The default parameters are used to determine the identity for the polypeptides of the present disclosure.

By way of example, a polypeptide sequence may be identical to the reference sequence, that is be 100% identical, or it may include up to a certain integer number of amino acid alterations as compared to the reference sequence such that the percent identity is less than 100%. Such alterations are selected from: at least one amino acid deletion, substitution (including conservative and non-conservative substitution), or insertion, and wherein said alterations may occur at the amino- or carboxy-terminus positions of the reference polypeptide sequence or anywhere between those terminal positions, interspersed either individually among the amino acids in the reference sequence, or in one or more contiguous groups within the reference sequence. The number of amino acid alterations for a given percent identity is determined by multiplying the total number of amino acids in the reference polypeptide by the numerical percent of the respective percent identity (divided by 100) and then subtracting that product from said total number of amino acids in the reference polypeptide.

The term “immunogenic amount” as used herein refers to an amount capable of eliciting the production of antibodies directed against the virus in the host to which the vaccine has been administered.

The term “immunogenic carrier” as used herein refers to a composition enhancing the immunogenicity of the virosomes from any of the viruses discussed herein. Such carriers include, but are not limited to, proteins and polysaccharides, and microspheres formulated using, for example, a biodegradable polymer such as DL-lactide-coglycolide, liposomes, and bacterial cells and membranes. Protein carriers may be joined to the proteinases, or peptides derived therefrom, to form fusion proteins by recombinant or synthetic techniques or by chemical coupling. Useful carriers and ways of coupling such carriers to polypeptide antigens are known in the art.

The term “immunogenic composition” as used herein refers to a composition that comprises an antigenic molecule where administration of the composition to a subject results in the development in the subject of a humoral and/or a cellular immune response to the antigenic molecule of interest.

The term “immunogenic fragment” as used herein refers to a fragment of an immunogen that includes one or more epitopes and thus can modulate an immune response or can act as an adjuvant for a co-administered antigen. Such fragments can be identified using any number of epitope mapping techniques, well known in the art (see, e.g., Epitope Mapping Protocols in Methods in Molecular Biology, Vol. 66 (Morris, G. E., Ed., 1996) Humana Press, Totowa, N.J.).

Immunogenic fragments can be at least about 2 amino acids in length, more preferably about 5 amino acids in length, and most preferably at least about 10 to about 15 amino acids in length. There is no critical upper limit to the length of the fragment, which can comprise nearly the full-length of the protein sequence or even a fusion protein comprising two or more epitopes.

The term “immunoglobulin” as used herein refers to a class of proteins that exhibit antibody activity and bind to other molecules (e.g., antigens and certain cell-surface receptors) with a high degree of specificity. Immunoglobulins can be divided into five classes: IgM, IgG, IgA, IgD, and IgE. IgG is the most abundant antibody class in the body and assumes a twisted “Y” shape configuration. With the exception of the IgMs, immunoglobulins are composed of four peptide chains that are linked by intrachain and interchain disulfide bonds. IgGs are composed of two polypeptide heavy chains (H chains) and two polypeptide light chains (L chains) that are coupled by non-covalent disulfide bonds.

The light and heavy chains of immunoglobulin molecules are composed of constant regions and variable regions. For example, the light chains of an IgG1 molecule each contain a variable domain (V_{L}) and a constant domain (C_{L}). The heavy chains each have four domains: an amino terminal variable domain (V_{H}), followed by three constant domains (C_{H}1, C_{H}2, and the carboxy terminal C_{H}3). A hinge region corresponds to a flexible junction between the C_{H}1 and C C_{H}2 domains. Papain digestion of an intact IgG molecule results in proteolytic cleavage at the hinge and produces an Fc fragment that contains the C_{H}2 and C_{H}3 domains, as well as two identical Fab fragments that each contain a C_{H}1 C_{L}, V_{H}, and V_{L} domain. The Fc fragment has complement- and tissue-binding activity. The Fab fragments have antigen-binding activity

Immunoglobulin molecules can interact with other polypeptides through a cleft within the C_{H}2-C_{H}3 domain. This “C_{H}2-C_{H}3 cleft” typically includes the amino acids at positions 251-255 within the C_{H}2 domain and the amino acids at positions 424-436 within the C_{H}3 domain. As used herein, numbering is with respect to an intact IgG molecule as in Kabat et al. (Sequences of Proteins of Immunological Interest, 5^{th} ed., Public Health Service, U.S. Department of Health and Human Services, Bethesda, Md.). The corresponding amino acids in other immunoglobulin classes can be readily determined by those of ordinary skill in the art.

The Fc region can bind to a number of effector molecules and other proteins, including the cellular Fe Receptor that provides a link between the humoral immune response and cell-mediated effector systems (Hamano et al., (2000) J. Immunol. 164: 6113-6119; Coxon et al., (2001) Immunity 14: 693-704; Fossati et al., (2001) Eur. J. Clin. Invest. 31: 821-831). The Fcγ receptors are specific for IgG molecules, and include FcγRI, FcγRIla, FcγRIlb, and FcγRIII. These isotypes bind with differing affinities to monomeric and immune-complexed IgG.

The term “immunopotentiator,” as used herein, is intended to mean a substance that, when mixed with an immunogen, elicits a greater immune response than the immunogen alone. For example, an immunopotentiator can enhance immunogenicity and provide a superior immune response. An immunopotentiator can act, for example, by enhancing the expression of co-stimulators on macrophages and other antigen-presenting cells.

The term “immunological response” as used herein refers to the development in a subject of a humoral and/or a cellular immune response to an antigen present in the composition of interest. For purposes of the present disclosure, a “humoral immune response” refers to an immune response mediated by antibody molecules, while a “cellular immune response” is one mediated by T-lymphocytes and/or other white blood cells.

One aspect of cellular immunity involves an antigen-specific response by cytolytic T-cells (“CTL”s). CTLs have specificity for peptide antigens that are presented in association with proteins encoded by the major histocompatibility complex (MHC) and expressed on the surfaces of cells. CTLs help induce and promote the destruction of intracellular microbes or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cellular immune response” also refers to the production of cytokines, chemokines and other such molecules produced by activated T-cells and/or other white blood cells, including those derived from CD4+ and CD8+ T-cells. Hence, an immunological response may include one or more of the following effects: the production of antibodies by B-cells; and/or the activation of suppressor T-cells and/or γδ T-cells directed specifically to an antigen or antigens present in the composition or vaccine of interest. These responses may serve to neutralize infectivity, and/or mediate antibody-complement, or antibody dependent cell cytotoxicity (ADCC) to provide protection to an immunized host. Such responses can be determined using standard immunoassays and neutralization assays, well known in the art.

The term “ligand for FcRγ” as used herein refers to a ligand for the γ chain of Fc receptors and means a substance that specifically binds to the γ chain of Fc receptors.

The term “modify the level of gene expression” as used herein refers to generating a change, either a decrease or an increase in the amount of a transcriptional or translational product of a gene. The transcriptional product of a gene is herein intended to refer to a messenger RNA (mRNA) transcribed product of a gene and may be either a pre- or post-spliced mRNA. Alternatively, the term “modify the level of gene expression” may refer to a change in the amount of a protein, polypeptide or peptide generated by a cell as a consequence of interaction of an siRNA with the contents of a cell. For example, but not limiting, the amount of a polypeptide derived from a gene may be reduced if the corresponding mRNA species is subject to degradation as a result of association with an siRNA introduced into the cell.

The term “nucleic acid molecule” as used herein refers to DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule can be single-stranded or double-stranded, but advantageously is double-stranded DNA. An “isolated” nucleic acid molecule is one that is separated from other nucleic acid molecules that are present in the natural source of the nucleic acid. A “nucleoside” refers to a base linked to a sugar. The base may be adenine (A), guanine (G) (or its substitute, inosine (I)), cytosine (C), or thymine (T) (or its substitute, uracil (U)). The sugar may be ribose (the sugar of a natural nucleotide in RNA) or 2-deoxyribose (the sugar of a natural nucleotide in DNA). A “nucleotide” refers to a nucleoside linked to a single phosphate group.

The term “oligonucleotide” as used herein refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides may be chemically synthesized and may be used as primers or probes. Oligonucleotide means any nucleotide of more than 3 bases in length used to facilitate detection or identification of a target nucleic acid, including probes and primers.

The term “operably linked” as used herein refers to an arrangement of elements wherein the components so described are configured so as to perform their usual function. Thus, a given promoter operably linked to a coding sequence is capable of effecting the expression of the coding sequence when the proper enzymes are present. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence.

The term “particle-forming polypeptide” as used herein refers to a particular viral (e.g., from a lentivirus) protein is meant a full-length or near full-length viral protein, as well as a fragment thereof, or a viral protein with internal deletions, insertions or substitutions, which has the ability to form VLPs under conditions that favor VLP formation. Accordingly, the polypeptide may comprise the full-length sequence, fragments, truncated and partial sequences, as well as analogs and precursor forms of the reference molecule. Thus, the term includes natural variations of the specified polypeptide since variations in coat proteins often occur between viral isolates. Preferred substitutions are those which are conservative in nature, i.e., those substitutions that take place within a family of amino acids that are related in their side chains. Specifically, amino acids are generally divided into four families: (1) acidic: aspartate and glutamate; (2) basic: lysine, arginine, histidine; (3) non-polar: alanine, valine, leucine, isoleucine, proline, phenylalanine, methionine, tryptophan; and (4) uncharged polar: glycine, asparagine, glutamine, cysteine, serine threonine, tyrosine. Phenylalanine, tryptophan, and tyrosine are sometimes classified as aromatic amino acids.

The terms “pharmaceutically acceptable” or “pharmacologically acceptable” as used herein refer to a material that is not biologically or otherwise undesirable, i.e., the material may be administered to an individual in a formulation or composition without causing any undesirable biological effects or interacting in a deleterious manner with any of the components of the composition in which it is contained.

The term “phenotypically mixed” as used herein refers to a VLP having at least two different surface molecules (e.g., a surface envelope glycoproteins and an adjuvant molecule) incorporated into the VLP. A phenotypically mixed VLP of the disclosure may include, for example, a heterologous target antigen peptide that it is desired to generate an immune response against.

The term “Polymerase Chain Reaction” or “PCR” refers to a thermocyclic, polymerase-mediated, DNA amplification reaction. A PCR typically includes template molecules, oligonucleotide primers complementary to each strand of the template molecules, a thermostable DNA polymerase, and deoxyribonucleotides, and involves three distinct processes that are multiply repeated to effect the amplification of the original nucleic acid. The three processes (denaturation, hybridization, and primer extension) are often performed at distinct temperatures, and in distinct temporal steps. In many embodiments, however, the hybridization and primer extension processes can be performed concurrently. The nucleotide sample to be analyzed may be PCR amplification products provided using the rapid cycling techniques described in U.S. Pat. Nos. 6,569,672; 6,569,627; 6,562,298; 6,556,940; 6,569,672; 6,569,627; 6,562,298; 6,556,940; 6,489,112; 6,482,615; 6,472,156; 6,413,766; 6,387,621; 6,300,124; 6,270,723; 6,245,514; 6,232,079; 6,228,634; 6,218,193; 6,210,882; 6,197,520; 6,174,670; 6,132,996; 6,126,899; 6,124,138; 6,074,868; 6,036,923; 5,985,651; 5,958,763; 5,942,432; 5,935,522; 5,897,842; 5,882,918; 5,840,573; 5,795,784; 5,795,547; 5,785,926; 5,783,439; 5,736,106; 5,720,923; 5,720,406; 5,675,700; 5,616,301; 5,576,218 and 5,455,175, the disclosures of which are incorporated by reference in their entireties. Other methods of amplification include, without limitation, NASBR, SDA, 3SR, TSA and rolling circle replication. It is understood that, in any method for producing a polynucleotide containing given modified nucleotides, one or several polymerases or amplification methods may be used. The selection of optimal polymerization conditions depends on the application.

A “polymerase” is an enzyme that catalyzes the sequential addition of monomeric units to a polymeric chain, or links two or more monomeric units to initiate a polymeric chain. In advantageous embodiments of this invention, the “polymerase” will work by adding monomeric units whose identity is determined by, and which is complementary to, a template molecule of a specific sequence. For example, DNA polymerases such as DNA pol 1 and Taq polymerase add deoxyribonucleotides to the 3′ end of a polynucleotide chain in a template-dependent manner, thereby synthesizing a nucleic acid that is complementary to the template molecule. Polymerases may be used either to extend a primer once or repetitively or to amplify a polynucleotide by repetitive priming of two complementary strands using two primers.

The term “polynucleotide” as used herein refers to any polyribonucleotide or polydeoxribonucleotide, which may be unmodified RNA or DNA or modified RNA or DNA. Thus, for instance, polynucleotides as used herein refers to, among others, single- and double-stranded DNA, DNA that is a mixture of single- and double-stranded regions, single- and double-stranded RNA, and RNA that is a mixture of single- and double-stranded regions, hybrid molecules comprising DNA and RNA that may be single-stranded or, more typically, double-stranded or a mixture of single- and double-stranded regions. The terms “nucleic acid,” “nucleic acid sequence,” or “oligonucleotide” also encompass a polynucleotide as defined above.

The term “polynucleotide” includes DNAs or RNAs as described above that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein.

It will be appreciated that a great variety of modifications have been made to DNA and RNA that serve many useful purposes known to those of skill in the art. The term “polynucleotide” as it is employed herein embraces such chemically, enzymatically, or metabolically modified forms of polynucleotides, as well as the chemical forms of DNA and RNA characteristic of viruses and cells, including simple and complex cells, inter alia.

A polynucleotide sequence of the present disclosure may be identical to the reference sequence, that is be 100% identical, or it may include up to a certain integer number of nucleotide alterations as compared to the reference sequence. Such alterations are selected from the group including at least one nucleotide deletion, substitution, including transition and transversion, or insertion, and wherein said alterations may occur at the 5′ or 3′ terminus positions of the reference nucleotide sequence or anywhere between those terminus positions, interspersed either individually among the nucleotides in the reference sequence or in one or more contiguous groups within the reference sequence. The number of nucleotide alterations is determined by multiplying the total number of nucleotides in the reference nucleotide by the numerical percent of the respective percent identity (divided by 100) and subtracting that product from said total number of nucleotides in the reference nucleotide. Alterations of a polynucleotide sequence encoding the polypeptide may alter the polypeptide encoded by the polynucleotide following such alterations.

The term “polypeptide” includes proteins and fragments thereof. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

The term “primer” as used herein refers to an oligonucleotide, the sequence of at least a portion of which is complementary to a segment of a template DNA which is to be amplified or replicated. Typically primers are used in performing the polymerase chain reaction (PCR).

The term “recombinant” as used herein refers to a polynucleotide of genomic, cDNA, semisynthetic, or synthetic origin which, by virtue of its origin or manipulation (1) is not associated with all or a portion of the polynucleotide with which it is associated in nature; and/or (2) is linked to a polynucleotide other than that to which it is linked in nature. The term “recombinant” as used with respect to a protein or polypeptide means a polypeptide produced by expression of a recombinant polynucleotide. “Recombinant host cells,” “host cells,” “cells,” “cell lines,” “cell cultures,” and other such terms denoting eukaryotic cell lines cultured as unicellular entities, are used interchangeably and refer to cells which can be, or have been, used as recipients for recombinant vectors or other transfer DNA, and include the progeny of the original cell which has been transfected. It is understood that the progeny of a single parental cell may not necessarily be completely identical in morphology or in genomic or total DNA complement to the original parent, due to accidental or deliberate mutation. Progeny of the parental cell which are sufficiently similar to the parent to be characterized by the relevant property, such as the presence of a nucleotide sequence encoding a desired peptide, are included in the progeny intended by this definition and are covered by the above terms. Techniques for determining amino acid sequence “similarity” are well known in the art.

The term “similarity” as used herein refers to the exact amino acid to amino acid comparison of two or more polypeptides at the appropriate place, where amino acids are identical or possess similar chemical and/or physical properties such as charge or hydrophobicity. A “percent similarity” then can be determined between the compared polypeptide sequences. Techniques for determining nucleic acid and amino acid sequence identity also are well known in the art and include determining the nucleotide sequence of the mRNA for that gene (usually via a cDNA intermediate) and determining the amino acid sequence encoded thereby, and comparing this to a second amino acid sequence. In general, “identity” refers to an exact nucleotide to nucleotide or amino acid to amino acid correspondence of two polynucleotides or polypeptide sequences, respectively.

The term “substantially purified” as used herein refers to the isolation of a substance (compound, polynucleotide, protein, polypeptide, polypeptide composition) such that the substance comprises the majority percent of the sample in which it resides. Typically in a sample a substantially purified component comprises 50%, preferably 80%-85%, more preferably 90-95%, of the sample. Techniques for purifying polynucleotides and polypeptides of interest are well-known in the art and include, for example, ion-exchange chromatography, affinity chromatography and sedimentation according to density.

The term “transfection” as used herein refers to a process by which agents are introduced into a cell. The list of agents that can be transfected is large and includes, but is not limited to, siRNA, sense and/or anti-sense sequences, DNA encoding one or more genes and organized into an expression plasmid, proteins, protein fragments, and more. There are multiple methods for transfecting agents into a cell including, but not limited to, electroporation, calcium phosphate-based transfections, DEAE-dextran-based transfections, lipid-based transfections, molecular conjugate-based transfections (e.g., polylysine-DNA conjugates), microinjection and others.

The terms “treat,” “treating,” and “treatment” as used herein refer to an approach for obtaining beneficial or desired clinical results. For purposes of embodiments of this disclosure, beneficial or desired clinical results include, but are not limited to, alleviation of symptoms, diminishment of extent of disease, stabilization (e.g., not worsening) of disease or conditions, preventing spread of disease or conditions, delaying or slowing of disease progression or condition, amelioration or palliation of the disease state or condition, and remission (partial or total) whether detectable or undetectable. In addition, “treat,” “treating,” and “treatment” can also mean prolonging survival as compared to expected survival if not receiving treatment.

The term “vaccine” as used herein refers to an immunogenic amount of one or more virosomes, fragment(s), or subunit(s) thereof. Such vaccines can include one or more viral surface envelope glycoproteins and portions thereof, and adjuvant molecule and portions thereof on the surfaces of the virosomes, or in combination with another protein or other immunogen, such as one or more additional virus components naturally associated with viral particles or an epitopic peptide derived therefrom.

The immunogenic compositions and/or vaccines of the present disclosure may be formulated by any of the methods known in the art. They can be typically prepared as injectables or as formulations for intranasal administration, either as liquid solutions or suspensions. Solid forms suitable for solution in, or suspension in, liquid prior to injection or other administration may also be prepared. The preparation may also, for example, be emulsified, or the protein(s)/peptide(s) encapsulated in liposomes.

The active immunogenic ingredients are often mixed with excipients or carriers, which are pharmaceutically acceptable and compatible with the active ingredient. Suitable excipients include but are not limited to water, saline, dextrose, glycerol, ethanol, or the like and combinations thereof. The concentration of the immunogenic polypeptide in injectable, aerosol or nasal formulations is usually in the range of about 0.2 to 5 mg/ml. Similar dosages can be administered to other mucosal surfaces.

In addition, if desired, the vaccines may contain minor amounts of auxiliary substances such as wetting or emulsifying agents, pH buffering agents, and/or other agents, which enhance the effectiveness of the vaccine. Examples of agents which may be effective include, but are not limited to, aluminum hydroxide; N-acetyl-muramyl-L-threonyl-D-isoglutamine (thr-MDP); N-acetyl-nor-muramyl-L-alanyl-D-isoglutamine (CGP 11637, referred to as nor-MDP); N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE); and RIBI, which contains three components extracted from bacteria: monophosphoryl lipid A, trehalose dimycolate and cell wall skeleton (MPL+TDM+CWS) in a 2% squalene/Tween 80 emulsion. The effectiveness of the auxiliary substances may be determined by measuring the amount of antibodies (especially IgG, IgM or IgA) directed against the immunogen resulting from administration of the immunogen in vaccines which comprise the adjuvant in question. Additional formulations and modes of administration may also be used.

The immunogenic compositions and/or vaccines of the present disclosure can be administered in a manner compatible with the dosage formulation and in such amount and manner as will be prophylactically and/or therapeutically effective, according to what is known to the art. The quantity to be administered, which is generally in the range of about 1 to 1,000 micrograms of viral surface envelope glycoprotein per dose and/or adjuvant molecule per dose, more generally in the range of about 5 to 500 micrograms of glycoprotein per dose and/or adjuvant molecule per dose, depends on the nature of the antigen and/or adjuvant molecule, subject to be treated, the capacity of the host's immune system to synthesize antibodies, and the degree of protection desired. Precise amounts of the active ingredient required to be administered may depend on the judgment of the physician or veterinarian and may be peculiar to each individual, but such a determination is within the skill of such a practitioner.

The vaccine or immunogenic composition may be given in a single dose; two-dose schedule, for example, two to eight weeks apart; or a multi-dose schedule. A multi-dose schedule is one in which a primary course of vaccination may include 1 to 10 or more separate doses, followed by other doses administered at subsequent time intervals as required to maintain and/or reinforce the immune response (e.g., at 1 to 4 months for a second dose, and if needed, a subsequent dose(s) after several months). Humans (or other animals) immunized with the virosomes of the present disclosure are protected from infection by the cognate virus.

It should also be noted that the vaccine or immunogenic composition can be used to boost the immunization of a host having been previously treated with a different vaccine such as, but not limited to, DNA vaccine and a recombinant virus vaccine.

The term “variant” refers to a polypeptide or polynucleotide that differs from a reference polypeptide or polynucleotide, but retains essential properties. A typical variant of a polypeptide differs in amino acid sequence from another, reference polypeptide. Generally, differences are limited so that the sequences of the reference polypeptide and the variant are closely similar overall (homologous) and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring, such as an allelic variant, or it may be a variant that is not known to occur naturally.

Modifications and changes can be made in the structure of the polypeptides of this disclosure and still result in a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly where the biologically functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent, polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take one or more of the foregoing characteristics into consideration are well known to those of skill in the art and include, but are not limited to (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure thus contemplate functional or biological equivalents of a polypeptide as set forth above. In particular, embodiments of the polypeptides can include variants having about 50%, 60%, 70%, 80%, 90%, and 95% sequence identity to the polypeptide of interest. The term “substantially homologous” is used herein to denote polypeptides of the present disclosure having about 50%, about 60%, about 70%, about 80%, about 90%, and preferably about 95% sequence identity to the reference sequence. Percent sequence identity is determined by conventional methods as discussed above. In general, homologous polypeptides of the present disclosure are characterized as having one or more amino acid substitutions, deletions, and/or additions.

The term “vector” as used herein refers to a genetic unit (or replicon) to which or into which other DNA segments can be incorporated to effect replication, and optionally, expression of the attached segment. Examples include, but are not limited to, plasmids, cosmids, viruses, chromosomes and mini-chromosomes. Exemplary expression vectors include, but are not limited to, baculovirus vectors, modified vaccinia Ankara (MVA) vectors, plasmid DNA vectors, recombinant poxvirus vectors, bacterial vectors, recombinant baculovirus expression systems (BEVS), recombinant rhabdovirus vectors, recombinant alphavirus vectors, recombinant adenovirus expression systems, recombinant DNA expression vectors, and combinations thereof.

The term “virus-like particle” (VLP), as used herein, refers to non-replicating, non-infectious, self-assembling particles which have a similar physical appearance to virus particles and include pseudoviruses. Virus-like particles may lack or possess dysfunctional copies of certain genes of the wild-type virus, and this may result in the virus-like particle being incapable of some function which is characteristic of the wild-type virus, such as replication and/or cell-cell movement. VLPs devoid of viral nucleic acids are also contemplated. VLPs are generally composed of one or more viral proteins, such as, but not limited to those proteins referred to as capsid, coat, shell, surface, structural proteins (e.g., VP1, VP2), or particle-forming polypeptides derived from these proteins, including the proteins described herein. VLPs can form spontaneously upon recombinant expression of capsid proteins in an appropriate expression system. Methods for producing particular VLPs are known in the art. The presence of VLPs following recombinant expression of viral proteins can be detected using conventional techniques known in the art, such as by electron microscopy, biophysical characterization, and the like. For example, VLPs can be isolated by density gradient centrifugation and/or identified by characteristic density banding. Alternatively, cryoelectron microscopy can be performed on vitrified aqueous samples of the VLP preparation in question, and images can be recorded under appropriate exposure conditions.

### DESCRIPTION

The present disclosure provides novel compositions and methods for the activation of CD4 T cells, as well as CD8 cells, of a human or animal subject that would not otherwise be activated, or only at levels that are ineffective for the reduction of cancer cell populations in the subject. It has been found that although a vaccine directed against specific antigenic sites of a tumor or tumor cell may induce the activation of CD8 cells, there is a significant or total failure to induce the activation of CD4 cells. This differential activation, which occurs even when the antigen is delivered to the subject by such as a lentiviral vector, also represents a significant deficiency in the ability to harness the full capabilities of the immune system to target cancer cells.

The present disclosure now provides recombinant fusion polypeptides, and virus-like particles assembled therefrom, that include a first domain that is a self-assembling virus-like particle that is linked to a ligand of the Foy receptor. The recombinant polypeptides of the disclosure can further include a third domain that is a target antigen peptide desired to be presented to the immune system of the recipient animal or human subject. It is contemplated that the first domain can be any polypeptide that can self-assembly into virus-like particles. Alternatively, a preferred first domain may include a Hepatitis B surface antigen alone, or in addition to other viral proteins or fragments thereof that can self-assemble.

For example, but not intended to be limiting, the data show that if the lentivirus vector includes a peptide domain derived from a ligand of the Foy receptor, and in particular an immunoglobulin Fc region, the lentivirus VLPs can be ingested by the target cell, such as a dendritic cell, and presented in conjunction with the MHC II complex to CD4 and CD8 cells via the MHC I complex, resulting in the activation and enhancement of the activity of both types of cells. It has been shown that the VLPs of the present disclosure are able to activate CD4 T cells to be directed to a target antigen when the animal or human subject has been administered a vaccine to the antigen which itself is unable to provide CD4 T cell activation.

The present disclosure provides recombinant fusion polypeptides that include an antigenic region, typically, but not limited to, an antigen or epitopic site isolated from a targeted cancer cell, together with a modified lentivirus coat proteins and the FcγR-binding domain. The modified lentivirus coat proteins of the disclosure can further include a fragment of the hepatitis B surface antigen. Such recombinant fusion proteins are able, therefore, not only to induce the activation of CD8 T cells, but to concurrently activate CD4 T cells, leading to a directed and simultaneous attack on target cells such as those of a tumor. The recombinant fusion proteins of the disclosure retain the ability to form virus-like particles. When delivered to the subject, the FcγR-binding domain most usefully binds to the surface FcγRs of antigen-presenting cells, whereupon the VLPs are taken up by such cells by the process of endocytosis and ultimately fragmented. The fragments may then be presented via the MHC II complex to CD4 T cells. Such activated T cells are able to release cytokines, elevating the humoral immune response directed against the target antigen that had been included in the VLPs.

It is contemplated that the present disclosure also provides recombinant nucleic acids that may be inserted into any suitable expression vector for the expression of the recombinant fusion proteins herein disclosed. After delivery of the recombinant expression vector to a suitable recipient cell, the desired recombinant fusion proteins may be expressed. The methods of generally making a recombinant expression vector, transformation of the cell system, and expression, are well known to those having ordinary skill in the art. Exemplary methods are herein presented as are more generally applicable methods suitable for use in the manufacture and use of the compositions herein disclosed.

Such expressed polypeptides may be isolated by techniques well-known in the art and allowed to form virus-like particles that may be delivered to a subject having such as a tumor. The VLPs of the disclosure may be readily formulated with adjuvants and pharmaceutically acceptable carriers for the delivery of effective amounts to the subject.

Cancer vaccine research, thus far, demonstrates that the induction of CD8 T cell response alone has little correlation with success in achieving antitumor effect. The lack of antitumor effect may be in part due to the inability of most current cancer vaccines to simultaneously co-activate CD4 T cells. The results of this disclosure show that activation of endogenous CD4 T cells by lentivirus immunization can affect the tumor milieu and antitumor effect of cancer vaccines. The disclosure provides constructs and methods to accomplish this. Surprisingly, it has been found that strong activation of endogenous CD4 T cells and enhancement of CD8 responses can be achieved by tagging the antigen with an Fc fragment, which increased the antitumor effect of lentivirus immunization, including regression of established tumors. This potent antitumor effect of Fc-tagging in the lentivirus platform is associated with improved immunologic changes in the tumor milieu, from substantial increase of TIL number and conversion of the tumor milieu into a Th1/Tc1-like pro-inflammatory microenvironment to significant decrease of the Treg ratio. These favorable changes in the tumor microenvironment and remarkable antitumor effects of lentivirus immunization were dependent on the activation of endogenous CD4 T cells. IFNγ production play critical roles in the CD4 mediated immunologic changes in the tumor milieu. Thus, in addition to the well-recognized CD4 role in priming adaptive immune responses, the results indicate that activation of endogenous CD4 helps the effector phase of antitumor immunity by modulating the tumor milieu.

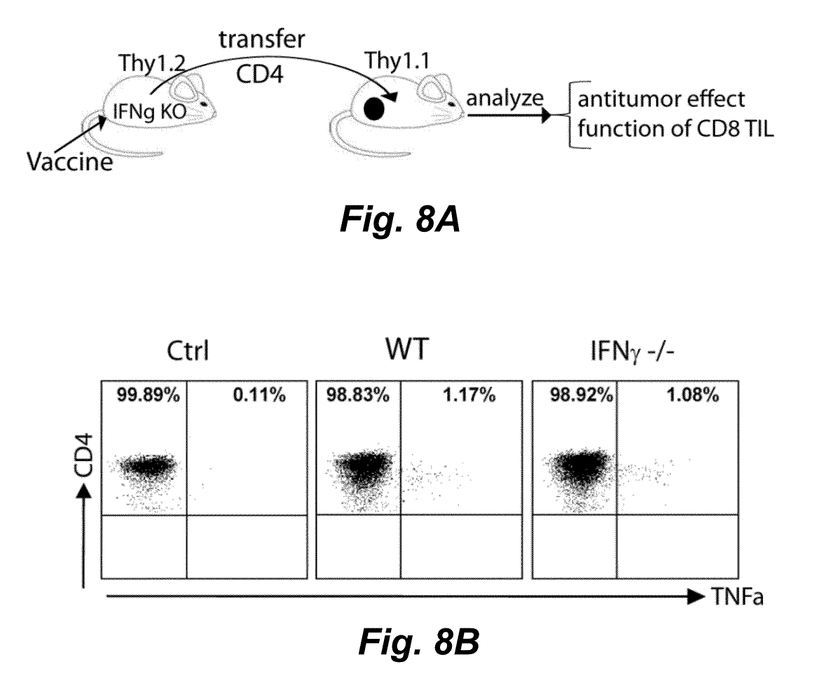

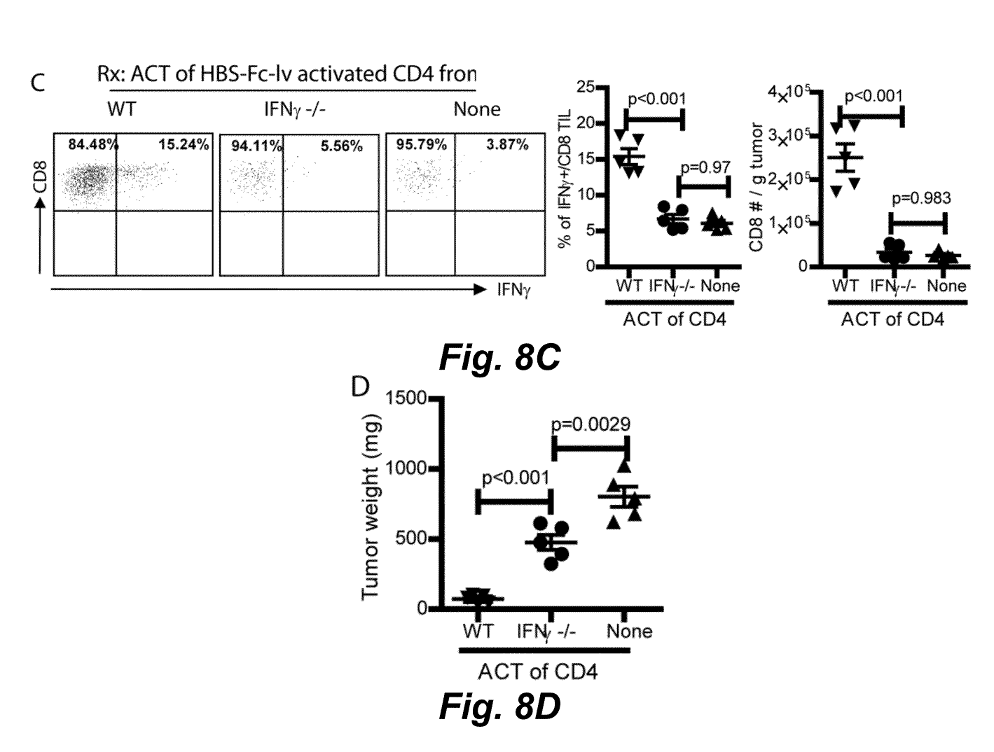

While not wishing to be bound by any one theory, activation of CD4 may help effector T cell migration into tumor lesions and license DCs to re-activate CD8 TIL. It has been found now that IFNγ production by activated CD4 T cells plays a critical role in mediating tumor infiltration of effector T cells into tumor lesions as the CD8 infiltration was markedly decreased after adoptive transfer of CD4 T cells from IFNγ knockout mice (as shown in FIG. 8C). These data show that adoptive transfer of TCR transgenic CD4 T cells can help migration of CD8 T cell to an infection site (Nakanishi et al., (2009) Nature 462: 510-513) and tumor lesion (Bos & Sherman (2010) Cancer Res. 70: 8368-8377) in an IFNγ-dependent manner. Therefore, it is likely that following activation by cancer vaccines, the activated endogenous CD4 T cells may enter tumors and secrete IFNγ to mediate chemokine production in the tumor stroma to attract more CD8 T cells. After migrating into tumor lesions, CD8 T cells, which are activated in the lymphoid tissues, need re-activation to execute their effector function in the tumor milieu. In this process, the CD40L on the activated CD4 T cells may license DCs to reactivate CD8 T cells in the tumor milieu to produce more IFNγ. The substantial increase of chemokines responsible for effector T cell recruitment into the tumor lesions after lentivirus immunization supports this argument. Then, the CD4 and CD8 TIL secrete more cytokines and turn the suppressive tumor lesion into a Th1/Tc1-like immune stimulatory microenvironment.

Activation of CD4 T cells can also reduce the Treg ratio in tumor lesions. Treg is a key component of immune suppression in tumor lesions (Yu et al., (2005) J. Exp. Med. 201: 779-791) and has been shown to inhibit effector function. It is also associated with poor clinical outcome (Zou, W. (2006) Nat. Rev. Immunol. 6: 295-307; Curiel et al., (2004) Nat. Med. 10: 942-949). The T effector/Treg ratio correlates with the outcome of tumor immunotherapy (Quezada et al. (2006) J. Clin. Invest. 116: 1935-1945; Quezada et al., (2008) J. Exp. Med. 205: 2125-2138). Thus, reduction of Treg can be an effective way to increase the antitumor efficacy of immunization (Hirschhorn-Cymerman et al., (2009) J. Exp. Med. 206: 1103-1116). What is still desired are effective methods of reducing the level of Treg. The present disclosure shows that activation of endogenous CD4 T cells by lentivirus immunization could markedly decrease the Treg ratio in tumor lesions. It is not clear how activation of endogenous CD4 T cells can decrease the Treg ratio in tumor lesions. But, two recent studies showed that Th1/Th2 T cells could inhibit peripheral Treg induction in vitro and in vivo (Wei et al., (2007) Proc. Natl. Acad. Sci. USA 104: 18169-18174) in an IFNγ-dependent manner (Caretto et al. (2010) J. Immunol. 184: 30-34). Addition of exogenous IFNγ markedly decreased Treg generation in vitro. Thus, while not limited to any one theory, it is possible that induction of endogenous CD4 T cells to become Th1 cells following HBS-Fc-Iv immunization reciprocally inhibits expansion of Treg. Alternatively, the induction of Treg apoptosis may be via the Fas-FasL pathway (Gritzapis et al., (2010) Cancer Res. 70: 2686-2696).

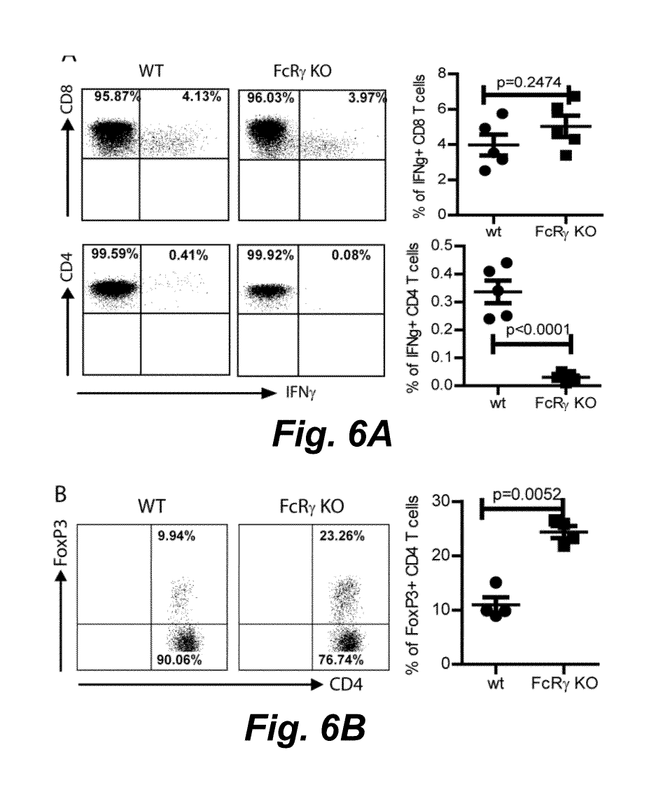

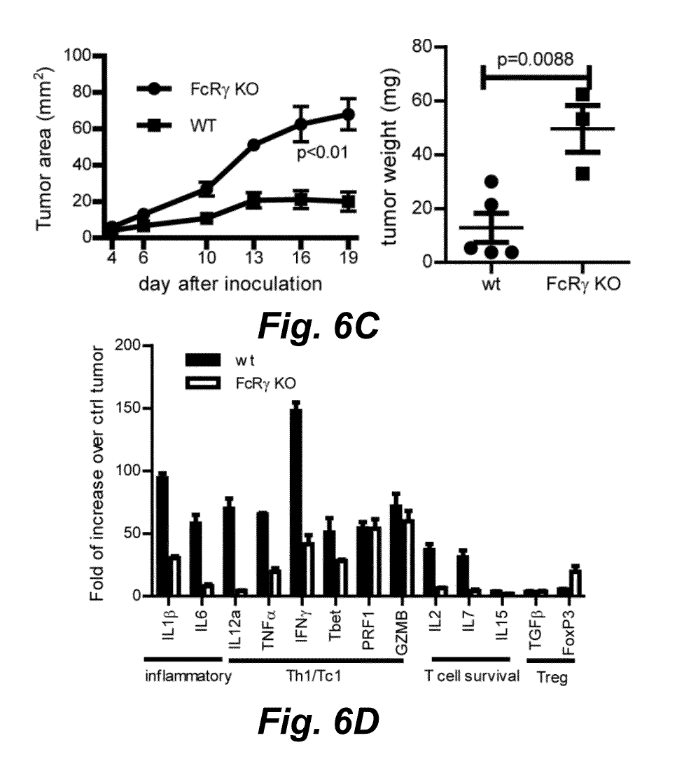

DCs have been found to take up Ab-Ag immune complexes efficiently via the FcγR to cross-prime CD8 T cells (Amigorena, S. (2002) J. Exp. Med. 195: F1-3; Dhodapkar et al. (2002) J. Exp. Med. 195: 125-133). However, even though covalent Fc tagging could significantly enhance CD8 T cell immune responses, FcγR may not play a critical role in lentivirus immunization since a similar magnitude of CD8 responses could be induced in FcRγ knockout mice (as shown in FIG. 6A). Following lentivirus immunization, direct presentation of antigen synthesized endogenously in the DCs is the main mechanism for priming CD8 T cells (He & Falo, Jr. (2007) Curr. Opin. Mol. Ther. 9: 439-446; He et al., (2006) Immunity 24: 643-656; He et al., (2007) Expert Rev. Vac. 6: 913-924). The increase of CD8 responses by Fc tagging may be mainly because of increase of antigen production (Hong et al. (2011) Vaccine 29: 3909-3916). The re-uptake of secreted antigen by DCs in an autocrine or paracrine fashion may only play a minor role in CD8 T cell activation. Thus, activation of CD8 T cells following lentivirus immunization is less dependent on FcγR-mediated cross-presentation.

In contrast, the results of the disclosure show that CD4 activation predominantly relies on the re-uptake of foreign antigen to enter the MHC II-mediated antigen processing and presentation pathway and thus depends on the FcγR-mediated antigen re-uptake.

To improve CD4 activation, various methods and antigens for activating endogenous CD4 T cells were studied and it was found that tagging HBsAg with immunoglobulin Fc fragment in the lentivirus immunization platform could potently activate endogenous CD4 T cells to produce IFNγ and to enhance CD8 responses (Hong et al., (2011) Vaccine 29: 3909-3916). The present disclosure addresses questions of if and how activation of endogenous CD4 T cells could be achieved to modulate the tumor milieu and to improve the antitumor effect of lentivirus immunization. The antitumor effect of lentivirus immunization and the immunologic changes in the tumor lesions with or without CD4 co-activation was studied. Immunization with lentivirus expressing Fc-tagged HBsAg activates both CD8 and CD4 responses and causes regression of established HBsAg+ B16 (B16-S) tumors. Immunological analysis revealed a significant increase of CD8 and CD4 T cells and preservation of their effector function in tumor lesions when CD4 cells were activated. The level of Th1/Tc-1-like cytokines and chemokines was also markedly increased in the tumor lesions in the presence of CD4 activation. On the other hand, the Treg ratio was substantially decreased in the immunized tumor when CD4 cells were activated. These favorable immunologic changes in the tumor lesions following lentivirus immunization were dependent on CD4 activation, which was mediated by Fcγ receptor (FcγR). Using an adoptive transfer approach, it was shown that the vaccine-activated CD4 T cells could effectively activate endogenous CD8 T cells in an IFNγ-dependent pathway. Accordingly, tagging tumor antigen with a Fc fragment according to the methods of the disclosure offers an effective way to activate endogenous CD4 and allows the activated CD4 T cells to improve the tumor milieu by increasing Th1/Tc1 like pro-inflammatory cytokines and chemokines, reducing the Treg ratio, and maintaining the effector function of TIL, which together result in an enhanced antitumor effect.

Utilizing HBsAg-expressing B16 tumor model has now shown that activation of endogenous CD4 T cells by active immunization can markedly relieve immune suppression in tumor lesions and substantially increase the antitumor effect. However, the expression of foreign HBsAg by the tumor cells per se in the absence of immunization does not reduce the Treg ratio in the tumor lesions, suggesting that expression of foreign antigen is not sufficient to significantly change the tumor microenvironment. More importantly, HBS-Iv immunization incapable of activating CD4 T cells did not significantly change the tumor milieu and resulted in no regression of B16-S tumor. Thus, the improvements of the tumor milieu, i.e., conversion of the tumor milieu into a Th1/Tc1-like environment and reduction of the Treg ratio in the tumor lesions by HBS-Fc-Iv immunization, are related to the vaccine's ability to activate CD4 T cells.

A number of viral vectors including modified vaccinia virus—(Hutchings et al., (2005) J. Immunol. 175: 599-606), measles virus—(Reyes-del Valle et al., (2009) J. ViroL 83: 9013-9017), and vesicular stomatitis virus—(Cobleigh et al., (2010) J. Virol. 84: 7513-7522) based vectors have been tested for inducing HBV-specific immune responses. Compared to protein-based vaccines, recombinant viral vectors have the advantages of effectively activating both cellular and humoral immune responses. Unlike most other viral vectors, lentivector encode no viral proteins except the desired transgene antigen. The immune responses can, therefore, be more focused on the intended antigen. Lentivector is also replication defective and thus can be safer. In addition, lentivector can effectively transduce dendritic cells, which directly prime naïve CD8 T cells for a prolonged period of time, possibly due to its lack of interference with the function of transduced dendritic cells (He et al., (2006) Immunity 24: 643-656; He et al., (2007) Expert Rev. Vaccines 6: 913-924). Lentivector immunization induce more potent immune responses (He et al., (2006) Immunity 24: 643-656) and long-lasting memory responses (Chapatte et al., (2006) Cancer Res. 66: 1155-1160).

The present disclosure provides evidence that HBS-Fc-Iv immunization can break immune tolerance in HBsAg^{low} Tg mice, generate HBsAg-specific immune responses, and result in seroconversion of HBsAg to anti-HBsAb. For example, animals with initial high viral load or chronic HBV patients with high viral titers may deplete the viral specific T cells during development in the thymus. However, it is also possible that regeneration of novel T cells from the thymus after viral load reduction may be able to replete the T cell repertoire with HBV-specific T cells and provide the capability of developing HBV-specific immunity in responding to immunization. Combination therapy of antiviral drugs and immunization that can break the immune tolerance in woodchuck hepatitis model provide supporting evidence for this theory (Menne et al., (2007) J. Virol. 81: 10614-10624).

The positive data from using HBsAg^{low} Tg mice, as provided in the present disclosure, suggests that it may be possible to generate HBV-specific immune responses with HBS-Fc-Iv immunization after the HBV viral load is decreased by antiviral drugs.

One aspect of the present disclosure, therefore, encompasses embodiments of a recombinant fusion protein for potentiating an immune system, the fusion protein comprising a self-assembling virus-like particle-forming polypeptide; an Fcγ receptor (FcγR)-binding ligand polypeptide, and a target antigen polypeptide, thereby forming a tripartite fusion polypeptide.

In the embodiments of this aspect of the disclosure, the target antigen polypeptide is positioned between the self-assembling virus-like particle-forming polypeptide and the Fcγ receptor (FcγR)-binding ligand polypeptide.

In the embodiments of this aspect of the disclosure, the self-assembling virus-like particle-forming polypeptide is selected from the group consisting of: an HPV L1 protein, an HPV L2 protein, an influenza Hemagglutinin A, an influenza neuraminidase, an influenza M1 protein, a lentivirus protein, any self-assembling fragment thereof, or any combination thereof.

In the embodiments of this aspect of the disclosure, the target antigen polypeptide is a hepatitis B surface antigen (HBsAg) polypeptide, or fragment thereof.

In the embodiments of this aspect of the disclosure, the immunoglobulin Fcγ receptor-binding ligand domain can comprise an immunoglobulin Fc domain.

In the embodiments of this aspect of the disclosure, the immunoglobulin Fcγ receptor-binding ligand domain can be isolated from an immunoglobulin G2a (IgG2a) or an immunoglobulin G1 (IgG1).

Another aspect of the disclosure encompasses embodiments of a nucleic acid encoding a recombinant fusion polypeptide as disclosed herein.

In the embodiments of this aspect of the disclosure, the nucleic acid can be inserted into an expression vector, wherein the nucleic acid is operably linked to an expression control region for expression of the nucleic acid in a recipient cell.

In the embodiments of this aspect of the disclosure, the nucleic acid can be within a host cell selected from a mammalian cell, an insect cell, a yeast cell, and a prokaryotic cell.

Another aspect of the disclosure encompasses embodiments of an immunopotentiating virus-like particle (VLP) comprising a recombinant fusion protein as disclosed herein.

Still another aspect of the disclosure encompasses embodiments of a method of generating a plurality of virus-like particles (VLP) comprising a plurality of recombinant fusion proteins, the method comprising: (a) providing a nucleic acid encoding a recombinant fusion protein according to the disclosure, where the nucleic acid is inserted into an expression vector, and where the nucleic acid is operably linked to an expression control region for expression the nucleic acid in a recipient cell; (b) delivering the nucleic acid to a host cell suitable for expression of the nucleic acid from the expression vector, where the host cell is selected from a mammalian cell, an insect cell, a yeast cell, and a prokaryotic cell; (c) expressing the nucleic acid in the host cell to provide said recombinant fusion protein; and (d) allowing the recombinant fusion protein to self-assemble to form a plurality of VLPs.

In the embodiments of this aspect of the disclosure, the method can further comprise the step of combining the plurality of VLPs with a physiologically acceptable carrier.

In the embodiments of this aspect of the disclosure, the method can further comprise the step of combining the plurality of VLPs with an adjuvant.

Still another aspect of the disclosure encompasses embodiments of an immunogenic composition comprising a plurality of immunopotentiating VLPs, wherein the immunopotentiating VLPs comprise a recombinant fusion protein as disclosed herein, wherein said immunogenic composition is devoid of an adjuvant.

Another aspect of the disclosure encompasses embodiments of an immunogenic composition comprising a plurality of immunopotentiating VLPs, where the immunopotentiating VLPs can comprise a recombinant fusion protein as disclosed herein, where the immunogenic composition can further comprise at least one adjuvant.

Another aspect of the disclosure encompasses embodiments of a method of inducing an immune response in an animal or human subject, comprising administering to the animal or human subject an immunogenic composition comprising a plurality of immunopotentiating VLPs comprising a recombinant fusion protein according to the present disclosure, whereby the VLPs are ingested by an antigen-presenting cell, thereby activating CD4 and CD8 T cells by MHC I and II.

In the embodiments of this aspect of the disclosure, the method can further comprise the step of allowing the activated CD4 and CD8 T cells to invade a tumor, thereby reducing the extent of the tumor in the recipient animal or human subject.

The specific examples below are to be construed as merely illustrative, and not limitative of the remainder of the disclosure in any way whatsoever. Without further elaboration, it is believed that one skilled in the art can, based on the description herein, utilize the present disclosure to its fullest extent. All publications recited herein are hereby incorporated by reference in their entirety.

It should be emphasized that the embodiments of the present disclosure, particularly, any “preferred” embodiments, are merely possible examples of the implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure and protected by the following claims.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### EXAMPLES

### Example 1

Cell lines, tumors, and mice: 293T cells were purchased from American Tissue and Cell Collection (ATCC, Manassas, Va.) and maintained in complete DMEM media. Fc receptor γ-chain (FcRγ) knockout mice were purchased from Taconic (Germantown, N.Y.). The HBsAg transgenic mice (C57BL/6J-Tg (Alb1HBV)44Bri/J) constitutively expressing HBsAg in the liver were purchased from Jackson Laboratory (Bar Harbor, Me.) and bred in the Laboratory Animal Services (LAS) of the Medical College of Georgia. C57BL/6 mice were obtained from either Taconic or the National Cancer Institute (Frederick, Md.). All mice were housed under SPF conditions and used at 6-10 weeks old.

To establish tumors, B16-S (5×10⁵) or B16-F10 (2×10⁵) cells were inoculated subcutaneously into the shaved flanks of C57BL/6 mice. Tumor growth was monitored by measuring the perpendicular diameters 3 times a week. The tumor mass was weighted at the end of experiments.

### Example 2

### Lentivectors and Immunization

Plasmid pRC/CMV-HBS (ayw) (Aldevron LLC, Fargo, N. Dak.). HBV small surface antigen (HBS) and HBS-IgG2a Fc fusion genes (HBS-Fc) were obtained by overlapping PCR using pRC/CMV-HBS and murine IgG2a as templates. The stop codon was deleted and the rest of the HBS gene was fused in frame to the Fc fragment gene so that HBS-Fc fusion antigen gene could be created. Sequences were verified. Recombinant lentivector plasmid was purchased from Invitrogen (San Diego, Calif.) and modified as previously reported (He et al., (2005) J. Immunol. 174: 3808-3817), incorporated herein by reference in its entirety.

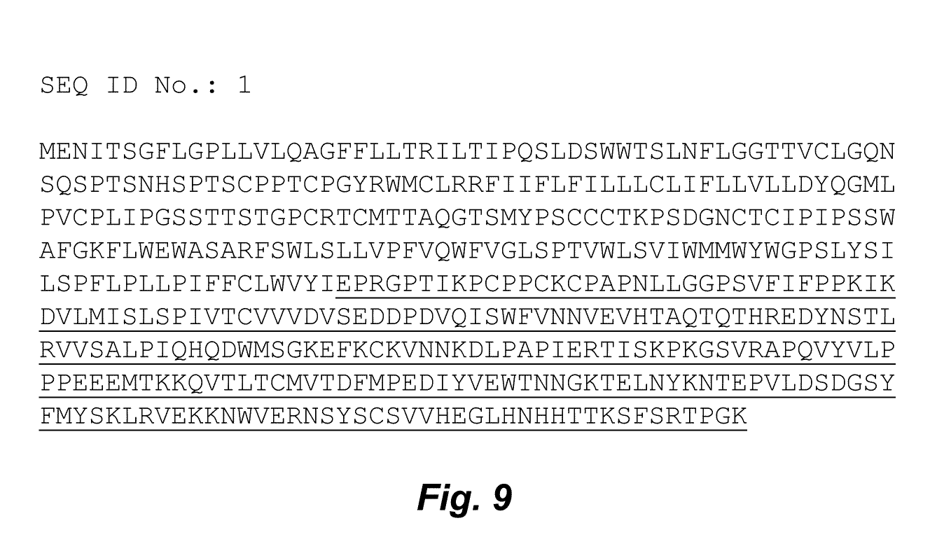

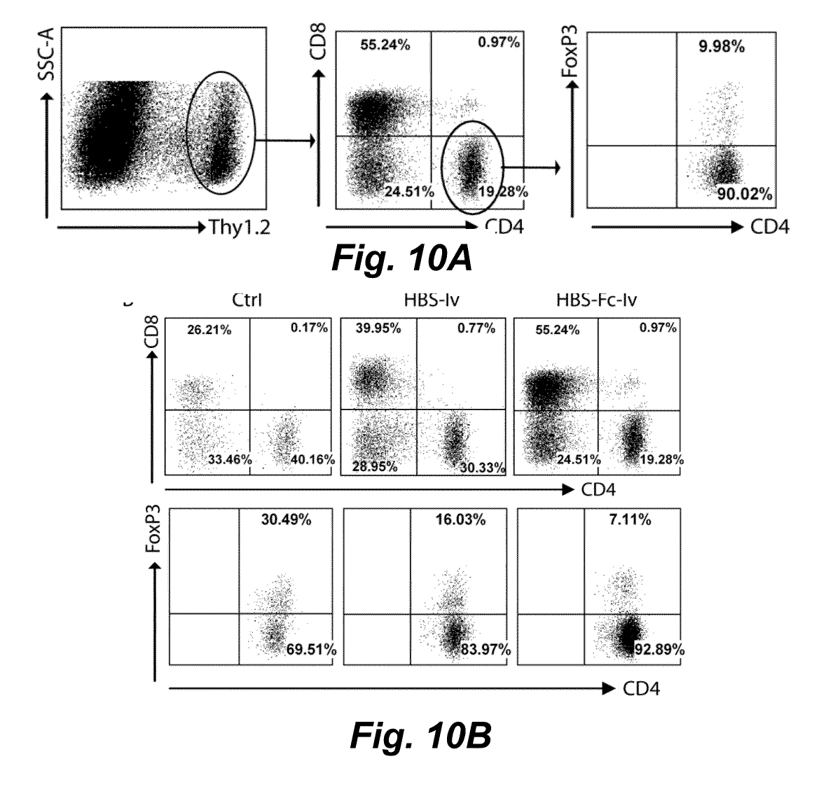

Lentivectors HBS-Iv (expressing the HBsAg without Fc tagging) and HBS-Fc-Iv (expressing the HBS-Fc fusion Ag) were constructed by replacing the TRP1 gene in TRP1-Iv (Liu et al., (2009) J. Immunol. 182: 5960-5969) with the small S full gene of HBV (ayw serotype) or the HBS-Fc fusion gene (HBS-Fc) that contained the HBsAg and CH2-CH3 domains (Fc fragment) of the mouse IgG 2a heavy chain. The entire amino acid sequence (SEQ ID No.: 1) of the HBS-Fc protein is shown in FIG. 9. Lentivirus preparation, concentration, and titration were conducted as described in (He et al., (2005) J. Immunol. 174: 3808-3817). For immunization, 2.5×10⁷ TUs of HBS-Iv or HBS-Fc-Iv were injected in the footpad. For tumor treatment, all immunizations were started on day 5, when the tumor lesions were clearly visible.

### Example 3

### Preparation of Single Cell Suspension from Tumor Lesions

The mice were sacrificed; tumors were collected and weighed. Tumor single-cell suspensions were prepared according to (Liu et al. (2009) J. Immunol. 182: 5960-5969). Briefly, 20-100 mg of each tumor was cut into small pieces and incubated at 37° C. for 0.5 h in RPMI containing 1 mg/ml of collagenase, 1 mg of hyaluronidase, and 100 units of DNase I. All enzymes were purchased from Sigma (St Louis, Mo.). Cells were then stained with various cocktails of indicated antibodies.

### Example 4

### Analysis of Tumor Infiltrating Lymphocytes by Flow Cytometry

The following antibodies used in this study, αCD45, αCD90, αCD8, αCD4, αCD40L, αCD107a, anti-Granzyme B (GrB), anti-FoxP3, anti-IFNγ, and anti-TNFα, were purchased from BD Biosciences (San Diego, Calif.), Biolegends (San Diego, Calif.), and eBioscience (San Diego, Calif.).

To measure cytokines, single cell suspensions from peripheral blood, spleen, or tumor were ex vivo stimulated for 4 hrs with 1 μg/ml of HBsAg peptide S₁₉₀₋₁₉₇ identified previously by Schirmbeck et al. ((2003) Eur. J. Immunol. 33: 3342-3352) (GenScript, Piscataway, N.J.) or 5 μg/ml of whole HBsAg (Propsec, East Brunswick, N.J.) in the presence of GolgiStop (BD Bioscience, San Diego, Calif.). In some experiments, the CD4 T cells were stimulated with PMA/Ionomycin (leukocyte activation cocktail, BD biosciences, San Diego, Calif.). Intracellular staining of IFN-γ and TNFα or Granzyme B was performed according to He et al., (2005) J. Immunol. 174: 3808-3817, incorpoprated herein by reference in its entirety. Alternatively, to measure degranulation, antibody against CD107a was added to the ex vivo cell culture, as described previously (Betts et al., (2003) J. Immunol. Meth. 281: 65-78). After staining, the cell events were collected using a FACScanto system (BD Bioscience, San Jose, Calif.). Data were analyzed using the FCS Express V3 software (De Novo Software, Ontario, Canada).

### Example 5

### Quantitative Reverse Transcription (qRT)-PCR

Tumor tissue total RNA was extracted using the RNA extraction kit from Qiagen (Valencia, Calif.). The expression level of chemokines was determined by using the Mouse Chemokines and Receptors RT² Profiler™ PCR Array (SAbioscience, Frederick, Md.) which can detect 89 different chemokines and receptors based on manufacturer's recommendation. Those chemokines with significant changes were further verified with qRT-PCR using primers derived from previous reports as described in (Martin et al. (2007) J. Immunol. 178: 4623-4631). To detect cytokine expression in the tumor lesions, qRT-PCR was also used. qRT-PCR to determine levels of FoxP3, Granzyme B, Perforin, TNFπ, and IFNγ were performed as described by Kohlmeyer et al. ((2009) Cancer Res. 69: 6265-6274). qRT-PCR primers for IL17, IL-21, RoRγt, and GADPH were derived from Das et al. ((2009) J. Exp. Med. 206: 2407-2416) and Tsujita et al. ((2006) Proc. Natl. Acad. Sci. USA 103: 11946-11951). qRT-PCR primers for other cytokines or transcription factors were derived from: T-bet (Xu et al., (2009) J. Immunol. 182: 6226-6236), TGFβ (Denning et al., (2007) J. Immunol. 178: 4230-4239), IL-6, IL-113 (Coste et al., (2007) Proc. Natl. Acad. Sci. USA 104: 13098-13103), IL-7 (Jeker et al. (2008) Blood 112: 3688-3695), IL12 (Blazquez & Berin (2008) J. Immunol. 180: 4441-4450), and IL-15 (McGill et al. (2010) J. Exp. Med. 207: 521-534). Primers for IL-2 are designed and synthesized as follows: IL-2 upstream: CCCTTGCTAATCACTCCTCA (SEQ ID NO.: 8); IL-2 downstream: GAGCTCCTGTAGGTCCATCA (SEQ ID NO.: 9).

### Example 6

### Adoptive Transfer

Wild type C57BL/6 mice (Thy1.1) or IFNγ knockout mice (Thy1.2 cogenic) were immunized with HBS-Fc-Iv or HBS-Iv. Two weeks after immunization, total CD8 and CD4 T cells were isolated using anti-CD4 and anti-CD8 magnetic microbeads as described by the manufacturer (Miltenyi Biotec, Auburn, Calif.). Purified T cells were then injected into the irradiated (low dose, 5Gy) mice bearing 5 day B16-S tumors.

### Example 7

### Statistical Analysis

Data were analyzed using student's unpaired t-test or ANOVA using the Prism software (GraphPad Prism, La Jolla, Calif.).

### Example 8

### Intracellular Staining of Cytokines

To measure cytokines, single-cell suspensions from peripheral blood were stimulated for 3 hrs with 1 μg/ml of HBS peptides (S₁₉₀₋₁₉₈) or overnight with recombinant HBsAg protein (5 μg/ml) with 10 μg/ml of hIL-2 (Prospec, Rehovot, Israel) in the presence of GolgiStop (BD Bioscience, San Diego, Calif.). To measure the cytokine production of liver-infiltrating T cells, the liver single cell suspension was enriched for T cells with 40% Percoll solution (GE-healthcare Bioscience AB, Uppsala, Sweden) after collagenase treatment as previously described (Zhou et al., (2004) J. Virol. 78: 3578-3600). Cells were then stimulated with peptides. Intracellular staining of IFN-γ was performed (He et al., (2005) J. Immunol. 174: 3808-3817). Surface staining included Thy1.2, CD4 and CD8. Cells were collected using a FACScanto system (BD Bioscience, San Jose, Calif.). Data were analyzed using FCS Express V3 software (De Novo Software, Ontario, Canada).

### Example 9

### In Vivo Killing Assay

To measure the cytolytic function of CD8 T cells, an in vivo killing assay was performed as described previously (He et al. (2005) J. Immunol. 174: 3808-3817; Barchet et al. (2000) Eur. J. Immunol. 30: 1356-1363). Briefly, HBS peptide-pulsed (targets) and non-pulsed (control) mouse splenocytes were labeled with 5 μM or 0.5 μM 5- (and -6)-carboxyfluorescein diacetate succinimidyl ester (CFSE), respectively, and then injected into mice. After 12 h, splenocytes were collected from mice and the specific lysis of target cells was examined and calculated (He et al., (2005) J. Immunol. 174: 3808-3817).

### Example 10

### ELISA

To compare the HBsAg expression in vitro, 293T cells were transduced with lentivector HBS-Iv or HBS-Fc-Iv. Supernatants (2.5 ml) from the transfected 293T cells were collected after 48 h and used directly for ELISA. To lyze the transduced 293T cells, 250 μl of Triton X-/SDS lysis buffer (50 mM Tris-HCI, pH 8.0, 150 mM NaCI, 0.1% SDS, 1% Triton X100, 1× protease inhibitor cocktail of BD Biosciences) was added. The cell lysates were then diluted with PBS (1:2) before measurement. To measure the HBsAg level, serum was collected from HBsAg Tg mice pre- and post-immunization. To detect HBsAg in the liver, liver samples (20 mg) were disaggregated and homogenized in 500 μl of Triton XSDS lysis buffer and the cell lysate was diluted with PBS (1:2). Then, HBsAg in the serum, cell lysate and supernatants, and liver samples were detected by using HBsAg ELISA kit according to the manufacturer's protocol (DiaSorin, Stillwater, Minn.). The anti-HBsAb in the serum was detected by using the anti-HBs detection kit from DiaSorin (Stillwater, Minn.).

### Example 11

### Detection of Serum Alanine Aminotransferase (sALT)

To examine the liver enzyme, mouse serum was collected from immunized or untreated control HBsAg^{low} Tg mice. The serum sALT was determined by using the ALT reagents from Teco Diagnostics (Anaheim, Calif.) according to the protocol of the manufacturer.

### Example 12

### Tagging the HBsAg with an Immunoglobulin Fc Fragment Markedly Increases the HBsAg Level in the Lentivector Transduced 293T Cells

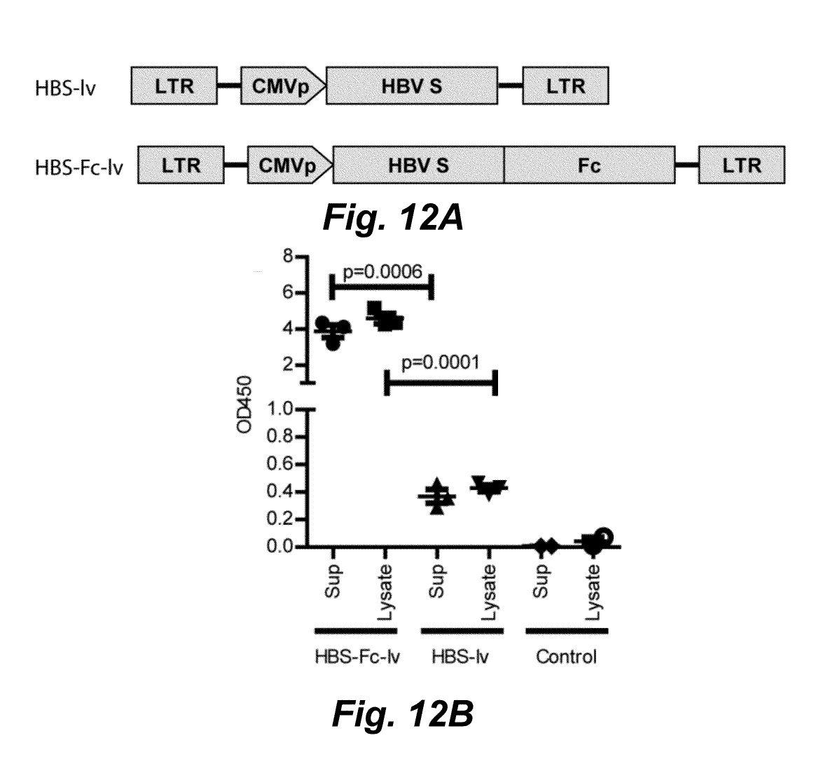

To investigate if lentivector could be utilized to stimulate HBV-specific immune responses, the HBV (type ayw) small S gene (HBs) was cloned into recombinant lentivector. Since immunoglobulin Fc fragment was found to enhance the immunization effect of plasmid DNA (You et al. (2001) Cancer Res. 61: 3704-3711), the HBsAg was tagged with mouse IgG2a Fc fragment. The two recombinant lentivectors were designated as HBS-Iv and HBS-Fc-Iv, respectively (as shown in FIG. 12A). To determine the expression and secretion of HBsAg and HBs-Fc fusion Ag, 293T cells were transduced with lentivector HBS-Iv or HBS-Fc-Iv. HBsAg in the supernatant and cell lysate were determined by ELISA. The level of HBsAg or HBS-Fc antigen was presented as the absolute amount of OD₄₅₀ in the supernatant or in the cell lysate. Fc fragment tagging at the C-terminal of protein increased the HBsAg level by 10-fold in both the supernatant and cell lysate of transduced 293T cells (as shown in FIG. 12B). The data resembled that of N-terminal fusion of Fc fragment that can increase the expression and secretion of protein in mammalian cells (Lo et al., (1998) Protein Eng. 11: 495-500). Fc tagging increases the availability of antigen and thus likely enhances the immune responses.

### Example 13

### Recombinant Lentivector Stimulates Potent HBsAg Specific CD8 T Cell Immune Responses

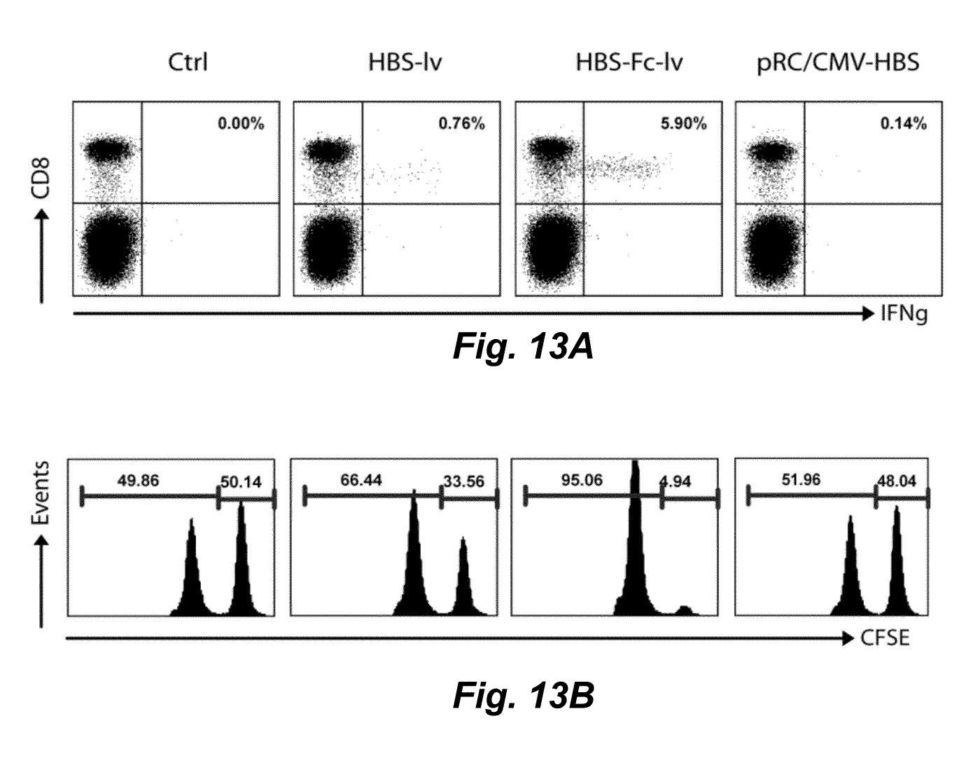

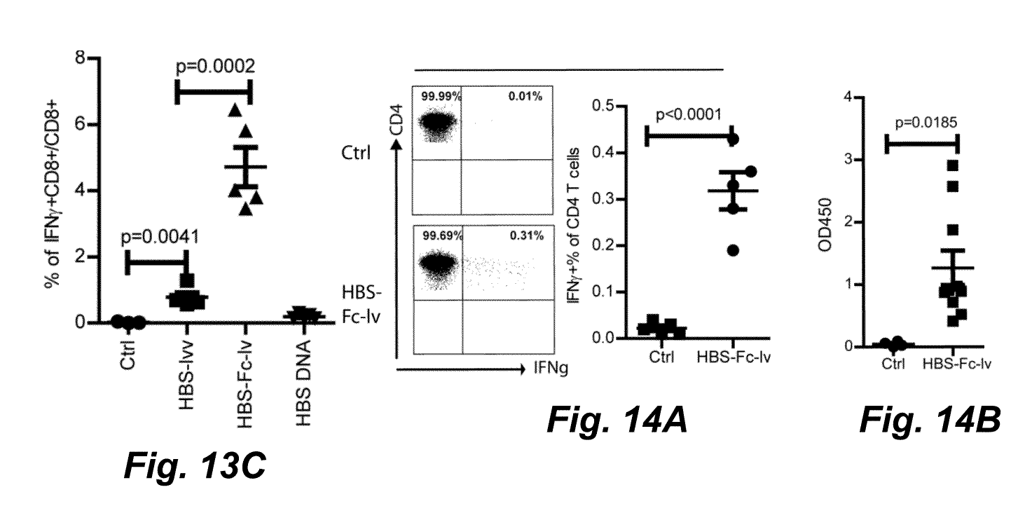

To study the efficacy of CD8 T cell responses stimulated by lentivectors and plasmid DNA immunization, C57BL/6 mice were immunized with either plasmid DNA or recombinant lentivectors HBS-Iv and HBS-Fc-Iv. CD8 T cell response was examined two weeks later. It was found that two injections of plasmid DNA elicited a weak immune response (0.1 to about 0.2% of CD8 T cells were IFNγ+). On the other hand, one injection of lentivector HBS-Iv induced moderate CD8 T cell responses with 0.5 to about 1% of CD8 cells being IFNγ+. Remarkably, one immunization with HBS-Fc-Iv expressing HBS-Fc fusion antigen stimulated the most potent T cell responses with about 6% of CD8 T cells producing IFNγ (as shown in FIGS. 13A and 13C). In addition, the in vivo killing data demonstrated that nearly all the HBS peptide (S₁₉₀₋₁₉₈) pulsed target cells were eliminated in the HBS-Fc-Iv immunized mice (FIG. 13B), demonstrating potent HBsAg-specific cytolytic function after HBS-Fc-Iv immunization. In contrast, only partial killing effect was found in HBS-Iv immunized mice and nearly no in vivo killing of target cells was detected in DNA-immunized mice. Results from both assays suggest that lentivector expressing HBs-Fc fusion antigen elicits much more potent CD8 T cell responses in the immunized C57BL/6 mice.

### Example 14

### Lentivector Immunization Also Induces CD4 T Cell Responses and Humoral Immune Responses

Lentivector is not effective in stimulating CD4 T cell responses if the antigen is synthesized and remains inside the cells (Rowe et al. (2006) Mol. Ther. 13: 310-319). Since HBS-Fc fusion antigen is a secretary protein and high level of antigen can be detected in the supernatant (FIGS. 13A-C), CD4 T cell responses can be induced after HBS-Fc-Iv immunization. Because no MHC II (I-A^{b}) restricted HBS epitope has been identified thus far, whole HBsAg recombinant protein was used for in vitro stimulation. Two weeks after immunization with HBS-Fc-Iv, using intracellular staining, IFNγ-producing CD4 T cells were detected after brief ex vivo HBsAg stimulation in the HBS-Fc-Iv immunized mice, suggesting that HBS-Fc-Iv immunization also stimulated CD4 T cell responses (as shown in FIG. 14A). On the other hand, immunization with HBS-Iv expressing HBsAg without Fc tagging did not stimulate measurable CD4 T cell responses.

To examine the humoral immune responses after HBS-Fc-Iv immunization, the anti-HBsAb in the serum of HBS-Fc-Iv immunized mice was determined. The data show that anti-HBsAb can be detected in all immunized mice even though a wide variation was observed among different animals (FIG. 14B).

Accordingly, the above data demonstrate that HBS-Fc-Iv lentivector immunization not only stimulates potent HBsAg-specific CD8 responses, but also CD4 responses and humoral responses in C57BL/6 mice, which is normally considered a low responder to HBV vaccines.

### Example 15

### Enhancement of CD4 and Humoral Responses, but not CD8 Responses after HBS-Fc-Iv Immunization is Mediated by Fc Receptor

One of the commonly cited mechanisms of how immunoglobulin Fc fragment enhance CD8 T cell responses is that Fc receptor on DCs can enhance antigen cross-presentation by taking up secreted antigen in the extracellular surrounding in a autocrine/paracrine fashion (Amigorena, S. (2002) J. Exp. Med. 195: F1-3). Ag-Ab complex can be taken up by DCs after binding Fc receptor and then be processed and presented via MHC I and II molecules. Ag-Ab protein complex has been shown to generate enhanced HBsAg specific immune responses in animal (Zheng et al., (2001) Vaccine 19: 4219-4225) and in human patients (Yao et al., (2007) Vaccine 25: 1771-1779) although no mechanisms were studied. To examine if the increase of CD8 T cell responses after HBS-Fc-Iv immunization was indeed through Fc receptor, wild type (WT) mice and Fc receptor γ-chain (FcRl) knockout (KO) mice were immunized with HBS-Fc-Iv. The CD8 T cell responses were examined two weeks following immunization.

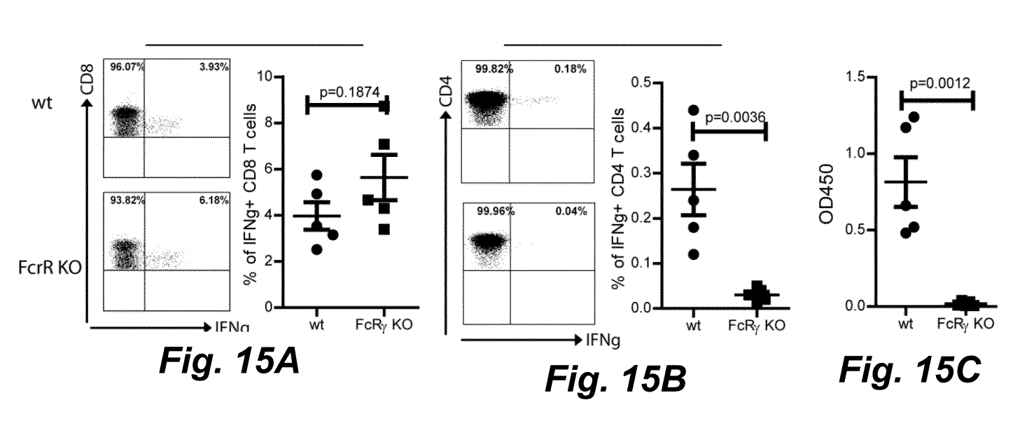

As shown in FIG. 15A, HBS-Fc-Iv immunization stimulated equally potent CD8 T cell responses in FcRγ knockout and wild-type mice, suggesting that the enhancement of the primary CD8 T cell responses by Fc fragment tagging is not mediated by Fc receptor. This is in accordance with the fact that the main pathway for CD8 T cell activation after lentivector immunization is via the MHC I molecule restricted presentation of endogenously synthesized protein by transduced skin DCs (He et al., (2006) Immunity 24: 643-656) and the Fc receptor-mediated cross-presentation may only play a minor role in activating CD8 T cell responses. In contrast, CD4 T cell response was severely inhibited in the FcRγ knockout mice (FIG. 15B). Because the MHC II restricted pathway is utilized mainly to process and present foreign (extracellular) antigen to activate CD4 T cells, it is likely that Fc receptor mediates foreign antigen uptake, processing, and presentation. Fc receptor knockout, thus, should mainly affect CD4 T cell responses.

While anti-HBsAb can be easily detected in wild-type mice, there is no anti-HBs Ab detected in the FcRγ knockout mice (FIG. 15C), suggesting that both CD4 and humoral immune responses induced by lentivector HBS-Fc-Iv immunization are mediated and affected by Fc receptor.

### Example 16

### Lentivector Immunization could Break Tolerance in HBsAg Tg Mice when the Serum Level of HBsAg is Low

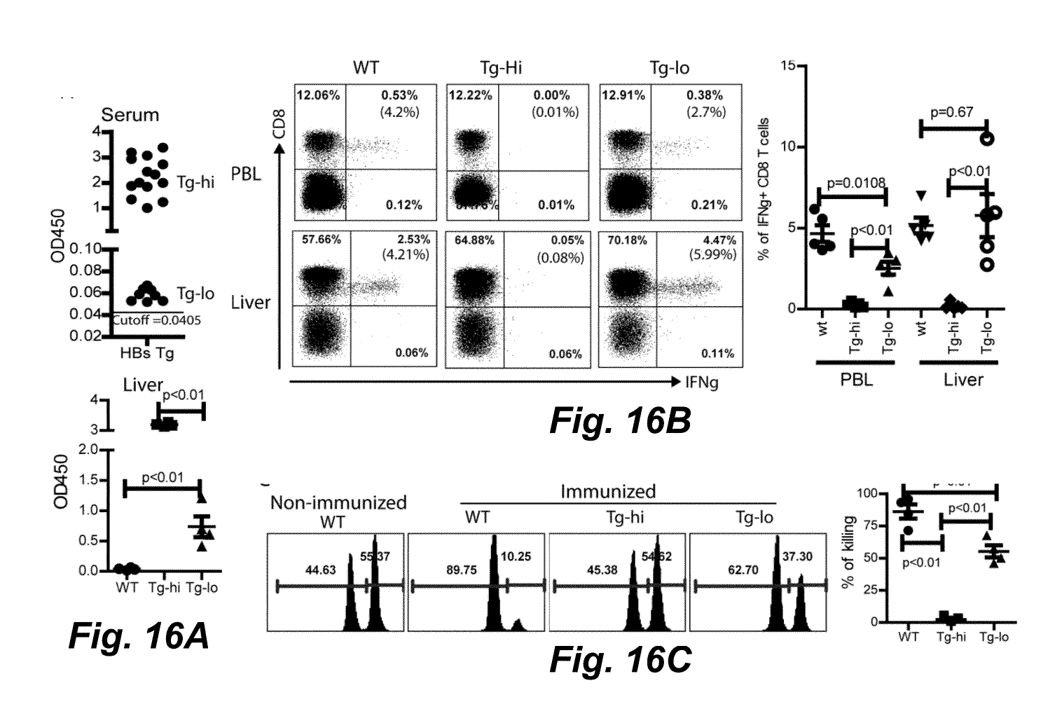

The above data has demonstrated that lentivector immunization could stimulate potent HBsAg-specific adaptive immune responses in naïve mice. However, for therapeutic purpose, lentivector immunization should be capable of eliciting HBsAg-specific immune responses in the presence of HBsAg, a situation that exists, for example, in chronic HBV infection. To examine if lentivector HBS-Fc-Iv immunization could break immune tolerance in the presence of HBsAg, the HBsAg Tg mice were used in which the expression of HBsAg is under the control of the albumin promoter in hepatocytes and there is a high level of HBsAg in the blood, mimicking the chronic HBV patients' conditions (Chisari et al., (1989) Cell 59: 1145-1156). When the HBsAg Tg mice were bred, two groups of offspring, HBsAg^{high} and HBsAg^{low} Tg mice, were observed (FIG. 16A, upper). To make sure that the HBsAg^{low} Tg mice are indeed expressing HBsAg, the HBsAg level in the liver tissue was examined. The liver of the HBsAg^{low} Tg mice contained a definitive level of HBsAg, on average an OD₄₅₀ of 0.75 (FIG. 16A, lower), confirming that HBsAg^{low} Tg mice were expressing HBsAg even though the level was significantly lower than that of HBsAg^{high} Tg mice.

Following HBS-Fc-Iv immunization, while potent HBsAg specific CD8 T cell responses were detected in wild-type mice, no HBsAg-specific CD8 T cell responses were induced in the HBsAg^{high} Tg mice. However, in the HBsAg^{low} Tg mice, HBS-Fc-Iv immunization elicited HBsAg-specific CD8 T cell responses that were detected, although the responses were lower compared to wild-type mice (FIG. 16B). A relatively higher percent of HBsAg-specific CD8 T cell response was detected in the liver compared to the peripheral blood of the Tg mice. This observation is consistent with previous findings that T cell infiltration and accumulation are antigen driven and dependent (Zhou et al. (2010) J. Immunol. 185: 5082-5092). To examine if the activated CD8 T cells in the HBsAg^{low} Tg mice maintain their cytolytic function, in vivo killing assay was conducted. There was no detectable in vivo killing activity in the HBsAg^{high} Tg mice. A significant in vivo killing activity could be detected in the HBsAg^{low} Tg mice even though at a significantly reduced level compared to wild-type mice. These data indicate that CD8 cytotoxic T cells maintain their target killing activity possibly at a reduced level.

### Example 17

### Lentivector HBS-Fc-iv Immunization Results in Seroconversion in the HBsAg^{low} Tg Mice

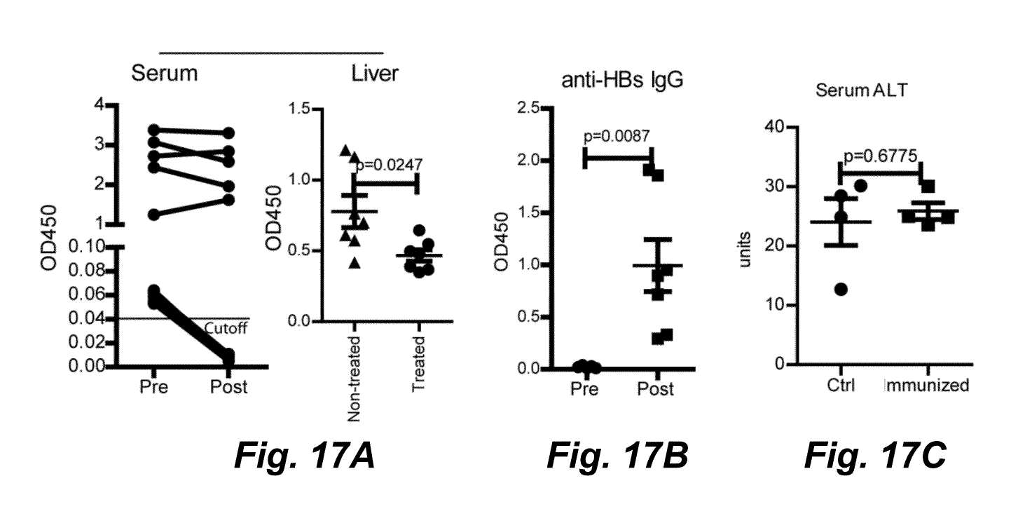

To study if the HBsAg-specific immune responses in the HBsAg^{low} Tg mice would result in clinical benefits, the change of HBsAg in the serum was examined. The HBsAg level in the serum of HBsAg^{low} Tg mice decreased to negative after HBS-Fc-Iv immunization (FIG. 17A, left). However, consistent with the absence of HBsAg-specific T cell responses, the HBsAg level remained unchanged in the HBsAg^{high} Tg mice following HBS-Fc-Iv immunization. Furthermore, lentivector immunization also decreased the HBsAg level in the liver of HBsAg^{low} Tg mice (FIG. 17A, right). Anti-HBsAb could also be easily detected in the serum (FIG. 17B), suggesting that seroconversion of HBsAg to anti-HBsAb occurred in the immunized HBsAg^{low} Tg mice. These data indicate that the lentivector HBS-Fc-Iv immunization can result in seroconversion of HBsAg to anti-HBsAb in HBsAg^{low} Tg mice.

Although in vivo CTL activity was detected (FIG. 16C) in HBsAg^{low} Tg mice, there was no obvious increase of the liver enzyme ALT in the serum (FIG. 17C), suggesting that there is not sufficient killing of HBsAg-expressing hepatocytes. Even though the data in FIG. 16B demonstrated that a high percent of the liver CD8 T cells was IFNγ+ after ex vivo stimulation with HBS peptides, the absolute number of T cells in the liver remained low.

The reduction of HBsAg level in the liver (FIG. 17A, right) is consistent with this idea and indicates that it is possible to achieve viral control without obvious lysis of hepatocytes. Therefore, even though not all of the HBV-expressing cells are eliminated by cytotoxic T lymphocytes, HBV viral replication may be suppressed or controlled by CD8 cytotoxic T lymphocytes and CD4 T cells in a non-cytolytic pathway.

### Example 18

### Fc Tagging Increases the CD8 as Well as CD4 Immune Responses of Iv Immunization that Causes Regression of Established Tumors

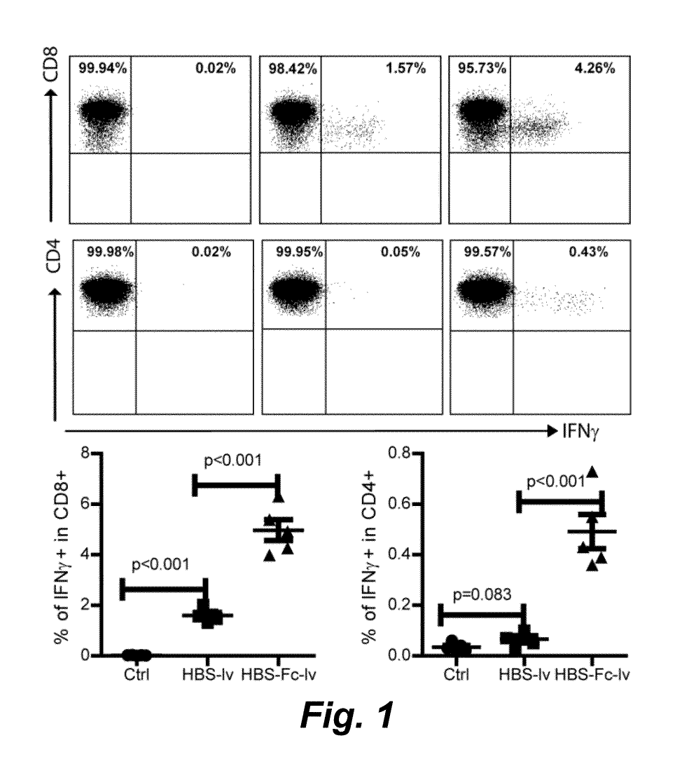

Lentivirus immunization is an effective approach to induce potent antigen specific CD8 responses (He et al., (2006) Immunity 24: 643-656; Liu et al., (2009) J. Immunol. 182: 5960-5969; He et al. (2007) Expert Rev. Vac. 6: 913-924; Esslinger et al., (2003) J. Clin. Invest. 111: 1673-1681) although activation of endogenous CD4 T cells by lentivirus immunization has proven difficult. However, lentivirus expressing the HBsAg fused with a Fc fragment (HBS-Fc fusion Ag) could effectively activate endogenous CD4 T cells in addition to enhancing CD8 responses (Hong et al., (2011) Vaccine 29: 3909-3916). To study if the Fc tag is indeed required to enhance the CD8 responses, and more importantly, to induce the activation of endogenous CD4 T cells, the magnitudes of CD8 and CD4 responses of HBS-Iv and HBS-Fc-Iv immunization were compared. Two weeks after immunization, peripheral blood cells were re-stimulated ex vivo with either HBS₁₉₀ peptide or whole HBsAg protein for 4 hrs before measuring the IFNγ level by intracellular staining. Compared to HBS-Iv, HBS-Fc-Iv immunization not only significantly increased the magnitude of CD8 responses, but also induced potent CD4 responses (FIG. 1). In contrast, HBS-Iv (without an Fc tag) immunization stimulated no measurable CD4 responses. Thus, tagging the lentivirus encoded antigen with Fc fragment induces the CD4 activation.

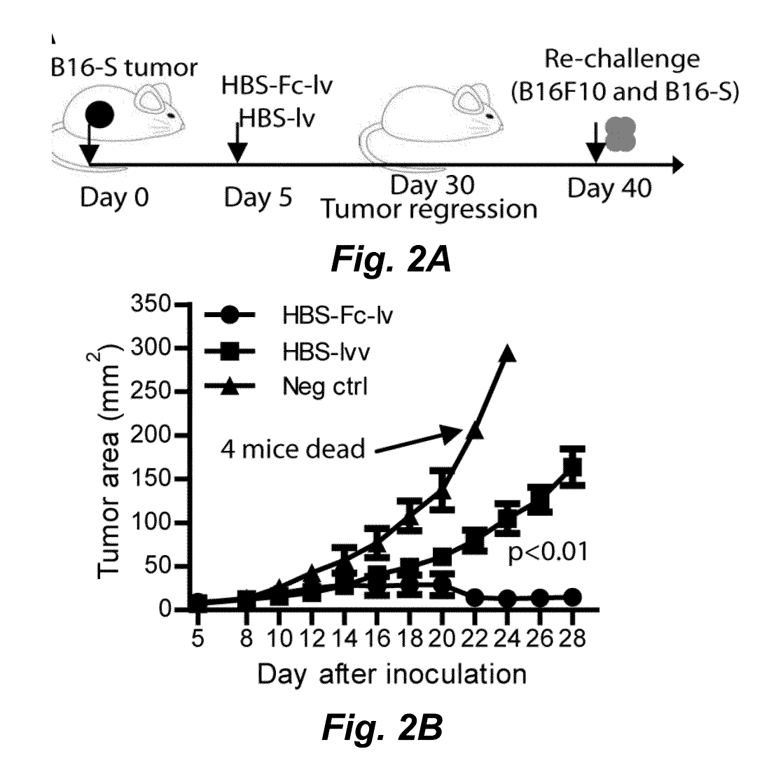

To study if the enhanced antigen-specific CD8 and CD4 immune responses are correlated with better antitumor effect of lentivirus immunization, mice bearing established B16-S tumors of sizes 10-15 mm² were treated with HBS-Fc-Iv or HBS-Iv immunization (FIG. 2A). As shown in FIG. 2B, compared to untreated controls, immunization with both HBS-Iv and HBS-Fc-lv could strongly inhibit B16-S tumor growth. However, only the tumors treated with HBS-Fc-Iv immunization experienced substantial regression and even complete eradication. During the peak of the immune response period, the majority of B16-S tumors in the group of mice treated with HBS-Fc-Iv underwent regression. Some of the tumors were completely eradicated, as shown in FIG. 2B. In a summary of 4 experiments, approximately 70-80% of well-established B16-S tumors experienced shrinkage after HBS-Fc-Iv immunization, and complete regression was found in 5 out of 20 tumor-bearing mice. The tumor-free mice from HBS-Fc-Iv treatment resisted further challenge by B16-S and B16-F10 tumor cells, indicating that the anti-tumor immune responses had spread to other tumor-associated antigens. In contrast, even though B16-S tumor growth was inhibited by HBS-Iv immunization, no tumor regression was observed. All mice in the HBS-Iv treated group eventually succumbed to tumor growth. In the lentivirus immunization platform, Fc tagging not only increased the magnitude of CD8 responses, but also induced potent CD4 responses, which may contribute to the tumor regression observed in HBS-Fc-Iv treated tumors.

### Example 19

### Fc Tagging Increases the Ability of Iv Immunization to Stimulate a Pro-Inflammatory Milieu within the Tumor Lesions

Tumor lesions are characterized as indolent chronic inflammation that can promote tumor growth (Grivennikov et al., (2010) Cell 140: 883-899). However, recent studies demonstrate that Th1 cytokines in tumor lesions may induce the chronic tumor promoting inflammation to becoming immune stimulating (Haabeth et al. (2011) Nat. Commun. 2: 240)

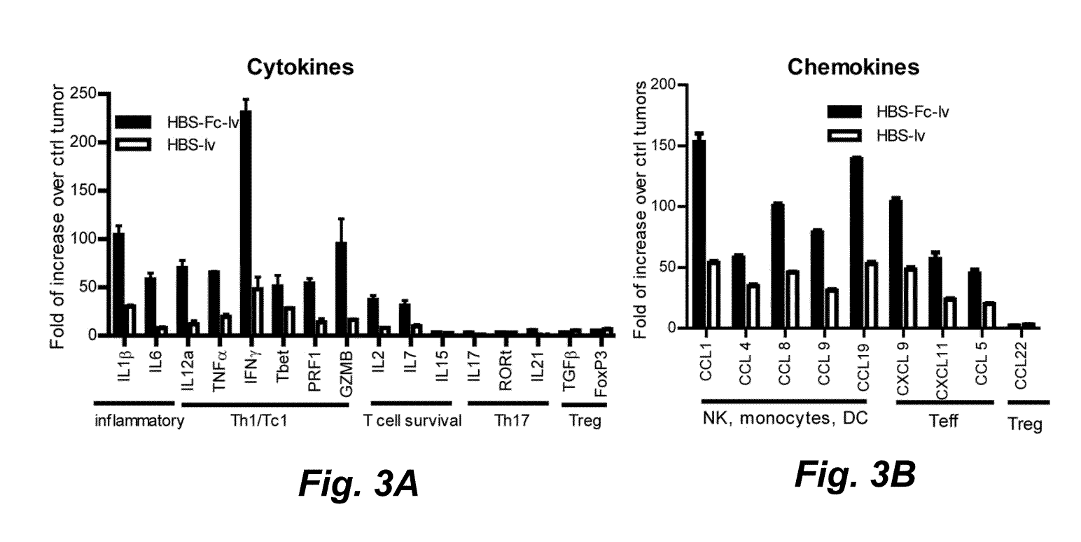

The regression of established tumors by HBS-Fc-Iv immunization provided a rationale for analyzing the inflammatory changes in tumor lesions. The levels of pro-inflammatory cytokines and chemokines were compared in tumor lesions after immunization with HBS-Iv and HBS-Fc-Iv using qRT-PCR. Compared to tumors without treatment, those that were treated with HBS-Fc-Iv had a 50- to 100-fold increase in mRNA levels of pro-inflammatory cytokines such as IL-1β and IL-6. At the same time, the Th1/Tc1 cytokines of IFNγ, perforin, granzyme, TNFα, and transcription factor T-bet in the tumor lesions of HBS-Fc-Iv immunized mice increased by 50- to 200-fold. In addition, cytokines for CD8 T cells survival such as IL-2 and IL-7 were significantly increased in the HBS-Fc-Iv immunized tumors. IL-15 was not obviously changed. Without Fc tagging, HBS-Iv immunization also increased the amount of pro-inflammatory and Th1/Tc cytokines in the tumor lesions but to a significant lesser extent. In contrast, the RNA levels of FoxP3, IL-17, and RORyt only increased slightly or remained unchanged, as shown in FIG. 3. The data demonstrate that Fc tagging significantly increases the effect of lentivirus immunization on converting tumor lesions into a Th1/Tc1-like immune stimulatory microenvironment.

Consistent with the Th1/Tc1-like cytokine changes, the chemokines responsible for attracting NK, DCs, Th1, and Tc1 cells in the tumor lesions of HBS-Fc-Iv-immunized mice increased as high as 150-fold compared to untreated tumor. Again, the lentivirus expressing the HBS-Fc fusion antigen demonstrated a much more significant effect (FIG. 3). In contrast, the chemokine CCL22 (Gobert et al. (2009) Cancer Res. 69: 2000-2009) for Treg recruitment was only slightly increased. These data indicate that HBS-Fc-Iv immunization alters chemokine levels such that innate immune effectors and T effectors, but not Tregs, can be effectively recruited into tumor lesions, which may play an important role in reshaping the tumor milieu to becoming less immune suppressive and more Th1/Tc1-like and immune stimulating.

### Example 20

Fc tagging markedly increases tumor infiltration of functional CD8 and CD4 T cells of lv immunization: The increase of chemokines in the tumor milieu following HBS-Fc-Iv immunization indicates that more T effectors could be recruited to the tumor lesions. The cellular immune components in the B16-S tumor lesions after different treatments were, therefore, tested.

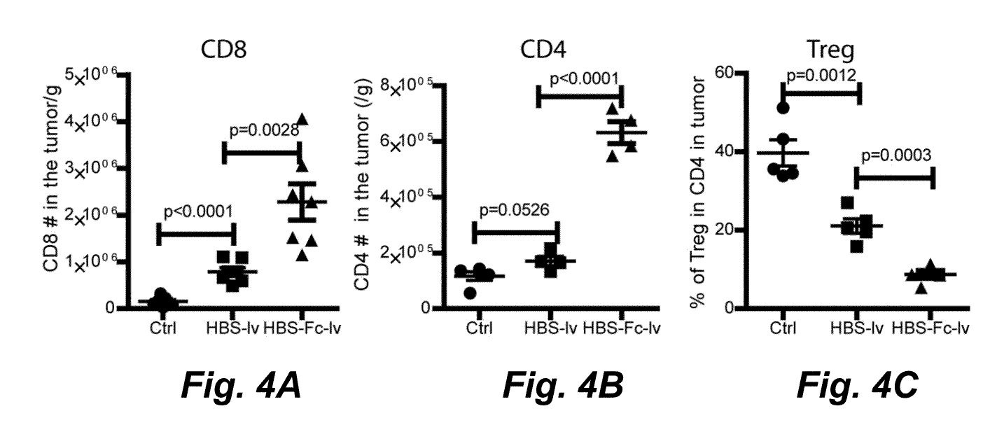

First, the absolute number of CD4 and CD8 TIL (as shown in FIGS. 4 and 8) were counted. HBS-Fc-Iv immunization induced significantly more CD8 T cell infiltration compared to HBS-Iv immunization (FIG. 4) and only HBS-Fc-Iv immunization significantly increased the number of CD4 TIL in the tumor lesions, consistent with data showing that only HBS-Fc-Iv immunization could effectively activate CD4 T cells (FIG. 1). HBS-Fc-Iv reduced the Treg ratio in tumor lesions more significantly than HBS-Iv immunization (FIGS. 4 and 8). Thus, in the lentivirus platform, Fc tagging significantly increases infiltration of CD4 and CD8 T cells into tumor lesions and at the same time reduces the Treg ratio. More specifically, the marked increase of CD4 TIL and reduction of Treg ratio were only observed in the HBS-Fc-Iv immunized tumor lesions.

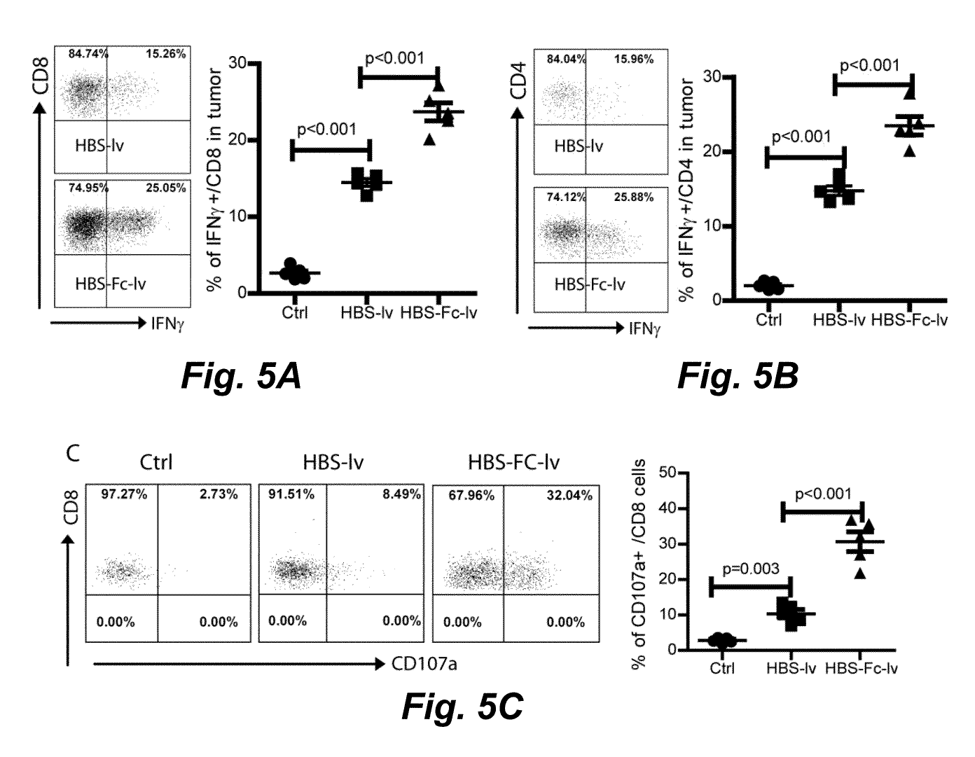

The effector function of CD8 and CD4 TIL following HBS-Fc-Iv and HBS-Iv immunization was also measured. As shown in FIG. 5, when compared to HBS-Iv immunization, HBS-Fc-Iv immunization stimulated more CD8 TIL to produce IFNγ in the tumor lesions (FIG. 5A). In addition, more CD4 TIL produced IFNγ in the tumors treated with HBS-Fc-Iv (FIG. 5B).

To examine the cytolytic function of CD8 TIL, cells were stained for CD107a, the degranulation marker in response to antigen stimulation that can be used as a surrogate test of cytolytic function (Betts et al., (2003) J. Immunol. Meth. 281: 65-78). Similar to the results of IFNγ staining, when compared to HBS-Iv immunization, HBS-Fc-Iv immunization caused more CD8 TIL to be CD107a positive (FIG. 5C). Because the absolute number (FIG. 4) and effector function (FIGS. 5A-5C) of CD8 and CD4 TIL were significantly increased in the HBS-Fc-Iv immunized tumors, the total number of functional CD8 and CD4 TILs in the HBS-Fc-Iv immunized tumors should be much higher. These data indicate that Fc tagging markedly increases the number and function of CD4 and CD8 effector T cells in the tumor lesions.

### Example 21

The immunologic changes in the tumor lesions and the antitumor effect of HBS-Fc-Iv immunization are dependent on CD4 activation: Using the two lentivirus (HBS-Iv and HBS-Fc-Iv) that could differentially activate CD4 T cells, it was shown that effective activation of CD4 T cells by HBS-Fc-Iv immunization may play an important role in increasing Th1/Tc1 like pro-inflammatory cytokines and functional effector T cell infiltration and decreasing Treg ratio in the tumor lesions. However, the differences in the magnitude of CD8 responses may also contribute to the immunologic changes in the tumor lesions.

FcRγ-knockout mice, because activation of CD4 T cells following HBS-Fc-Iv immunization was severely compromised while the CD8 response was not affected (Hong et al., (2011) Vaccine 29: 3909-3916) (FIG. 6A), allow study of the immunologic changes in the tumor milieu in the absence of CD4 activation. The data showed that following HBS-Fc-Iv immunization, the Treg ratio in the tumors of FcRγ-knockout mice was significantly higher than that in the wild-type mice (FIG. 6B). Furthermore, increase of Th1 cytokines in the tumor milieu after lentivirus immunization was significantly compromised in the FcRγ-knockout mice (FIG. 6C). Consistent with the FoxP3 staining data (FIG. 6B), the level of FoxP3 mRNA was significantly higher in the FcRγ-knockout tumor (FIG. 6C). Concurrently, the antitumor effect of HBS-Fc-Iv immunization in FcRγ knockout mice was also compromised (FIG. 6D). These data suggest that CD4 activation play an important role to increase the Th1/Tc1 cytokines and to decrease the Treg ratio in tumors and to enhance the antitumor effect of HBS-Fc-Iv immunization.

### Example 22

### Adoptive Transfer of CD4 T Cells can Activate Endogenous CD8 TIL in the Tumors and Generate Antitumor Effects

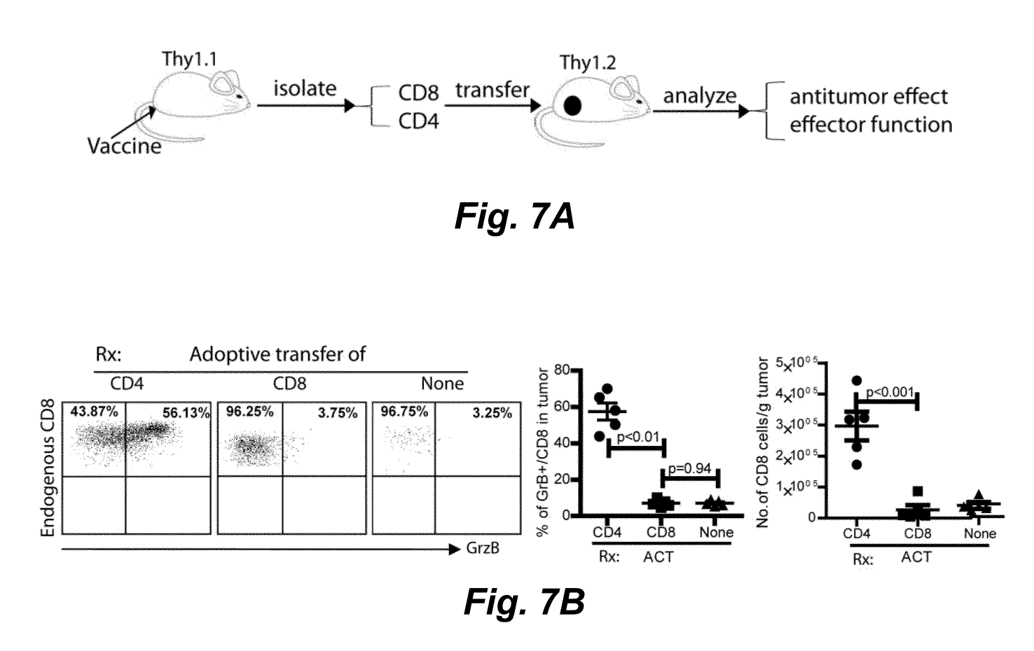

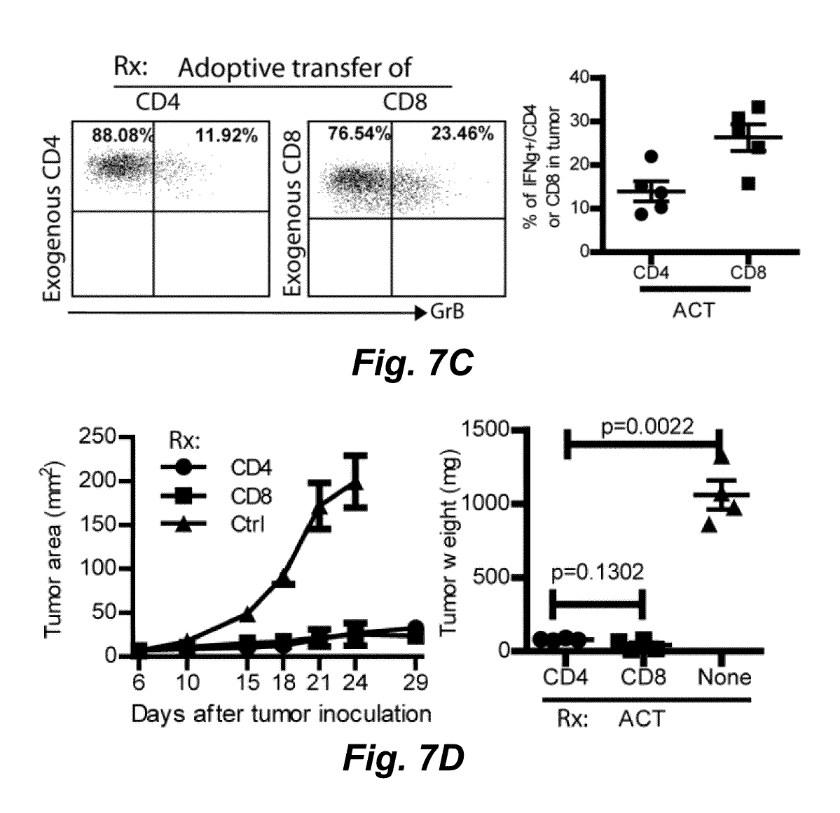

CD4 and CD8 T cells were isolated from HBS-Fc-Iv immunized Thy1.1 congenic C57BL/6 mice and adoptively transferred into irradiated B16-S tumor bearing Thy1.2 congenic mice (as schematically shown in FIG. 7A). Two weeks later, the effector function of TIL was analyzed. Adoptive transfer of activated CD4 T cells could activate endogenous CD8 TIL to express granzyme B (FIG. 7B) and IFNγ. In addition, more endogenous CD8 T cells were recruited into tumor lesions in the presence of activated CD4 T cells. In contrast, transfer of activated CD8 T cells did not increase granzyme expression of the endogenous CD8 TIL (FIG. 7B). The adopted exogenous CD8 as well as CD4 T cells were found to be capable of expressing granzyme B in the tumor lesions (FIG. 7C). Adoptive transfer of CD4 or CD8 T cells could effectively inhibit tumor growth, but low dose irradiation alone did not significantly affect tumor growth (FIG. 7D). Thus, adoptive transfer of both activated CD4 and CD8 T cells achieved similar antitumor effects in tumor bearing mice, but the mechanisms were different. The anti-tumor effect of CD4 was mediated indirectly by activating endogenous CD8 T cells, whereas the exogenous CD8 T cells may directly kill tumor cells.

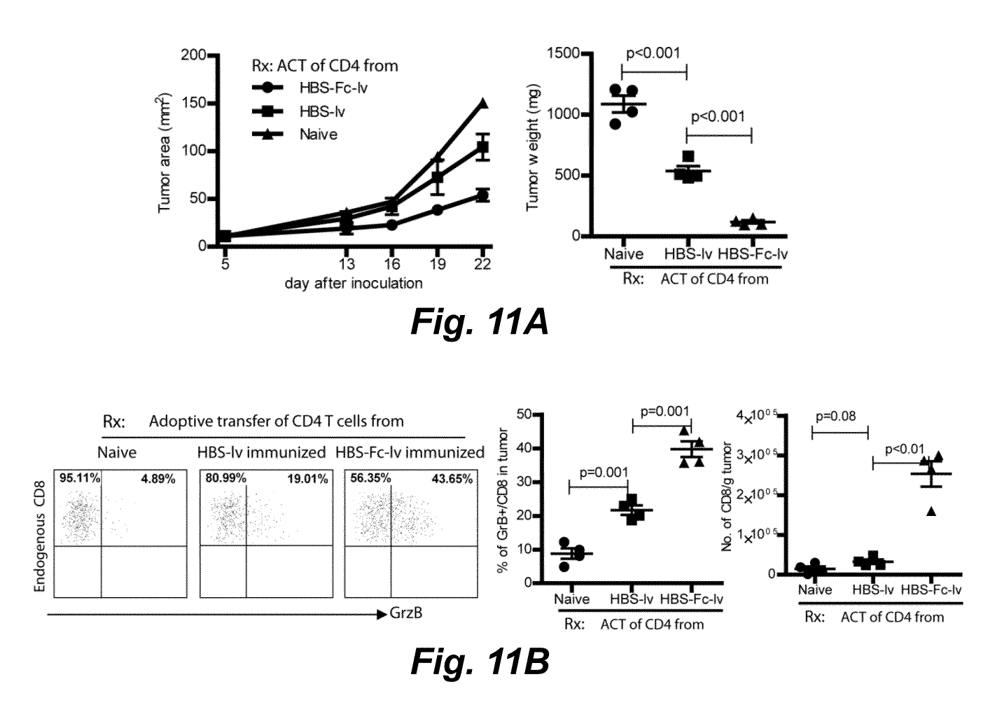

The antitumor activity of the CD4 T cells from mice immunized with HBS-Fc-Iv or HBS-Iv were compared as the level of CD4 activation was drastically different. Adoptive transfer of the pre-activated CD4 T cells from HBS-Fc-Iv immunized mice could result in more infiltration of endogenous CD8 TILs that possessed better effector function (FIG. 11), resulting in stronger antitumor effects (FIG. 11).

### Example 23

### IFNγ Expression by CD4 T Cells Plays a Critical Role in the CD4-Mediated Antitumor Effect

Using TCR Tg CD4 T cells, IFNγ produced by CD4 T cells played a critical role in helping recruit CD8 T cells to virally infected lesions (Nakanishi et al., (2009) Nature 462: 510-513) and tumor lesions (Bos & Sherman (2010) Cancer Res. 70: 8368-8377; Wong et al., (2008) J. Immunol. 180: 3122-3131). The antitumor effect of CD4 T cells from wild-type and IFNγ knockout mice was, therefore, examined. Wild-type and IFNγ knockout mice were immunized with HBS-Fc-Iv. Then, CD4 T cells from immunized wild-type and IFNγ knockout mice (Thy1.2) were isolated and adoptively transferred into tumor bearing mice (Thy1.1) to monitor their antitumor effects and their activation of endogenous CD8 T cells (FIG. 8A). Based on the TNFα expression, comparable numbers of activated CD4 T cells were found in the wild-type and IFNγ knockout mice after HBS-Fc-Iv immunization (FIG. 8B). The transfer experiment showed that, compared to wild-type CD4 T cells, adoptive transfer of CD4 T cells from the IFNγ knockout mice was incapable of activating endogenous CD8 T cells in the tumor lesions (FIG. 8C). Furthermore, tumor infiltration of endogenous CD8 T cells was also significantly decreased in mice treated with adoptive transfer of CD4 T cells from IFNγ knockout mice (FIG. 8C). Thus, adoptive transfer of CD4 T cells from IFNγ knockout mice severely compromised antitumor effect (FIG. 8D). Expression of IFNγ by vaccine-induced CD4 T cells plays a critical role in activating endogenous CD8 TIL and in mediating antitumor effect.

### Example 24

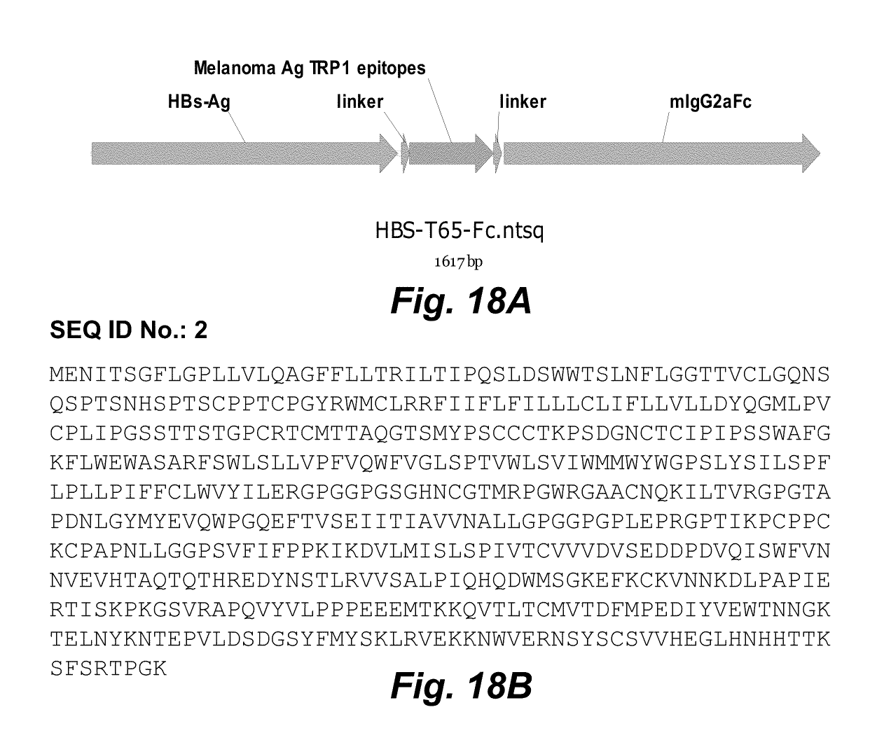

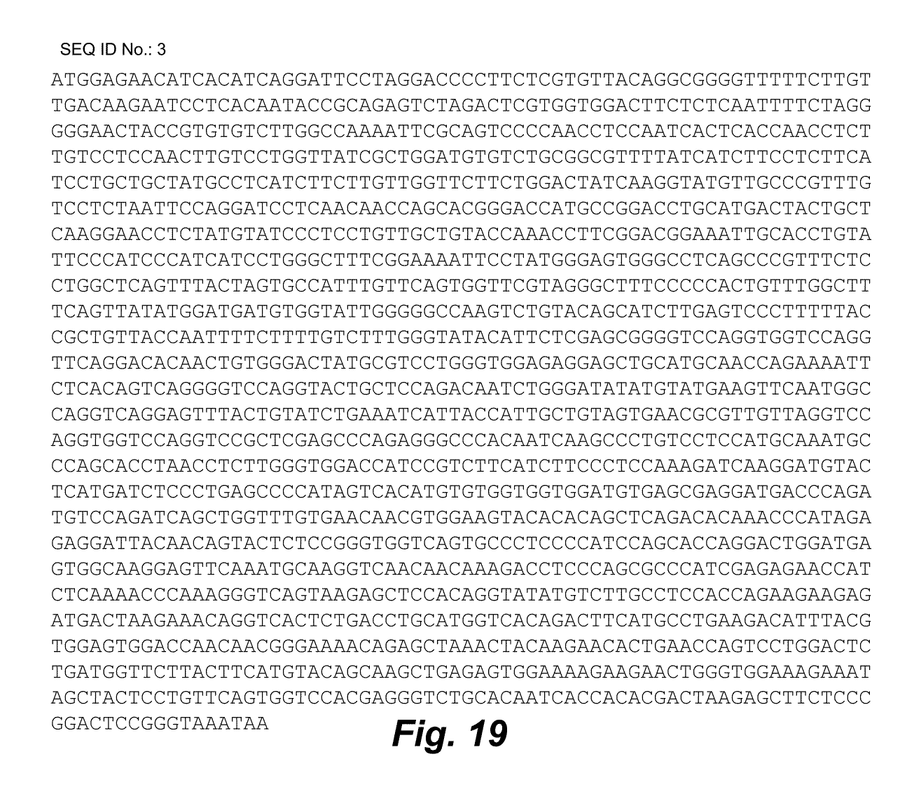

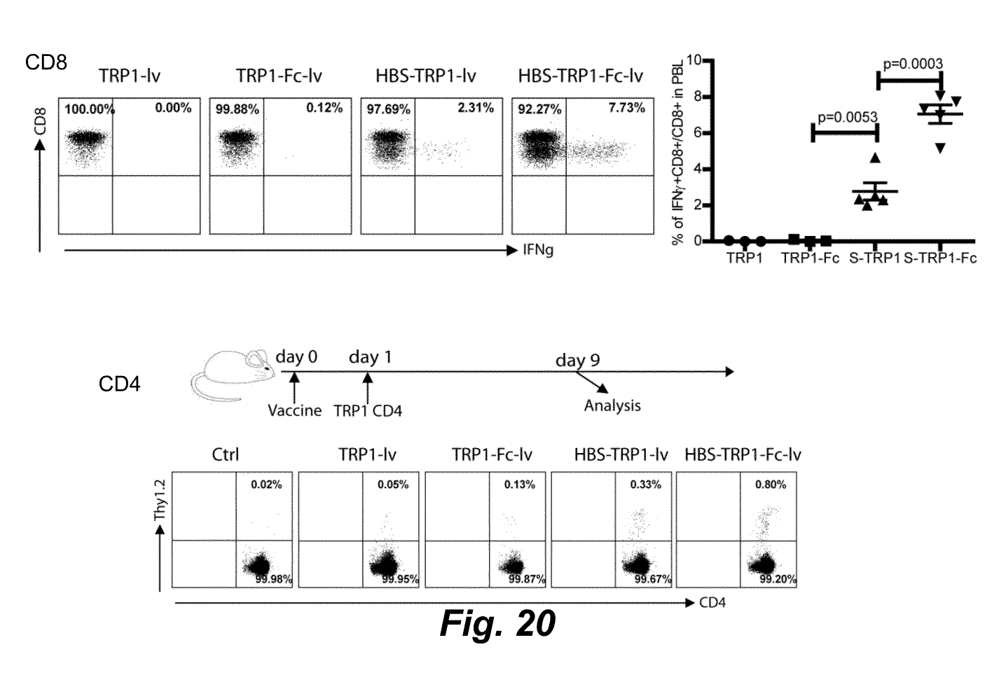

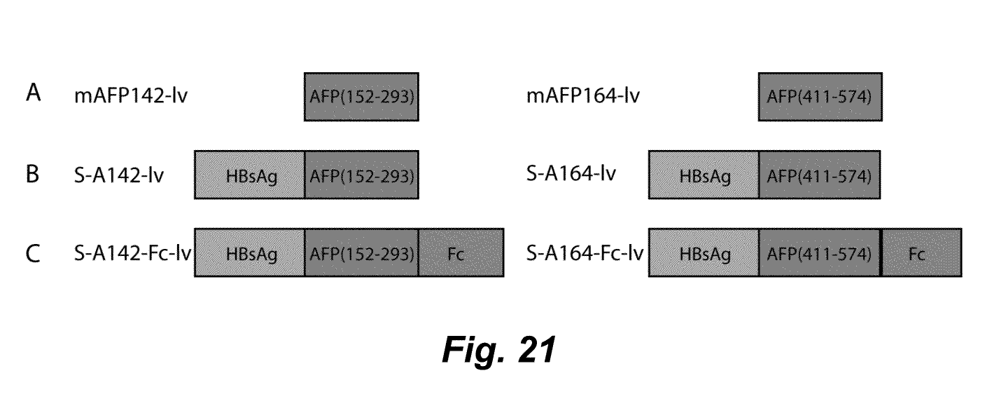

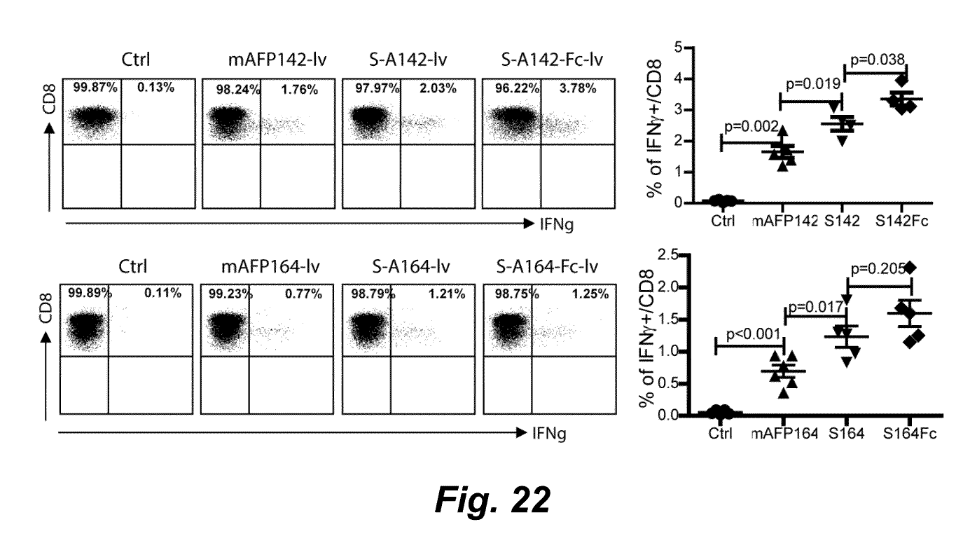

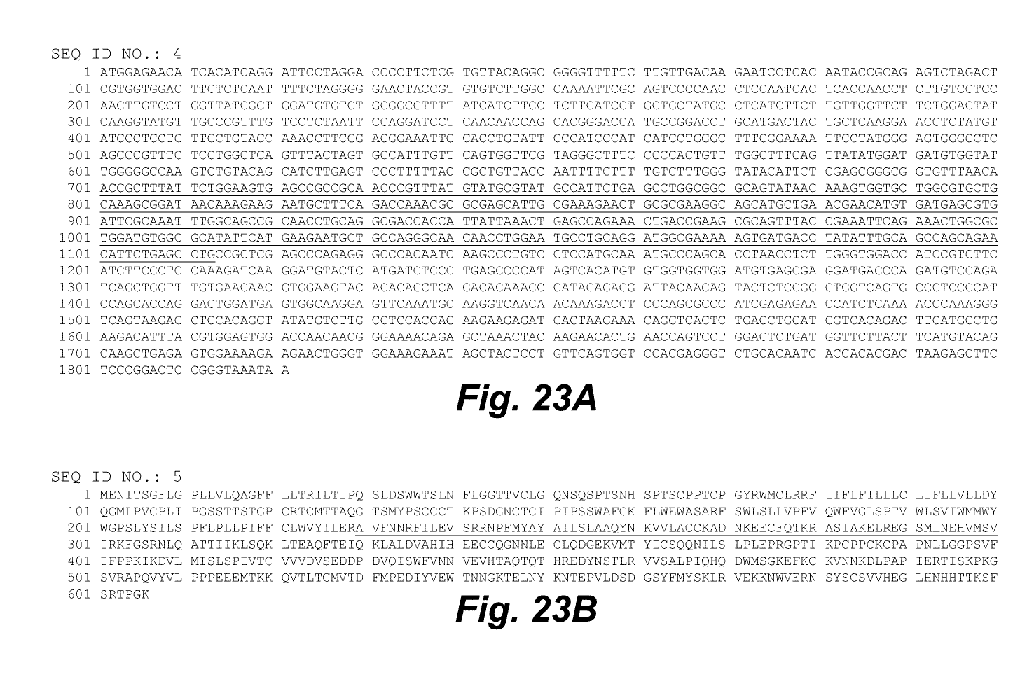

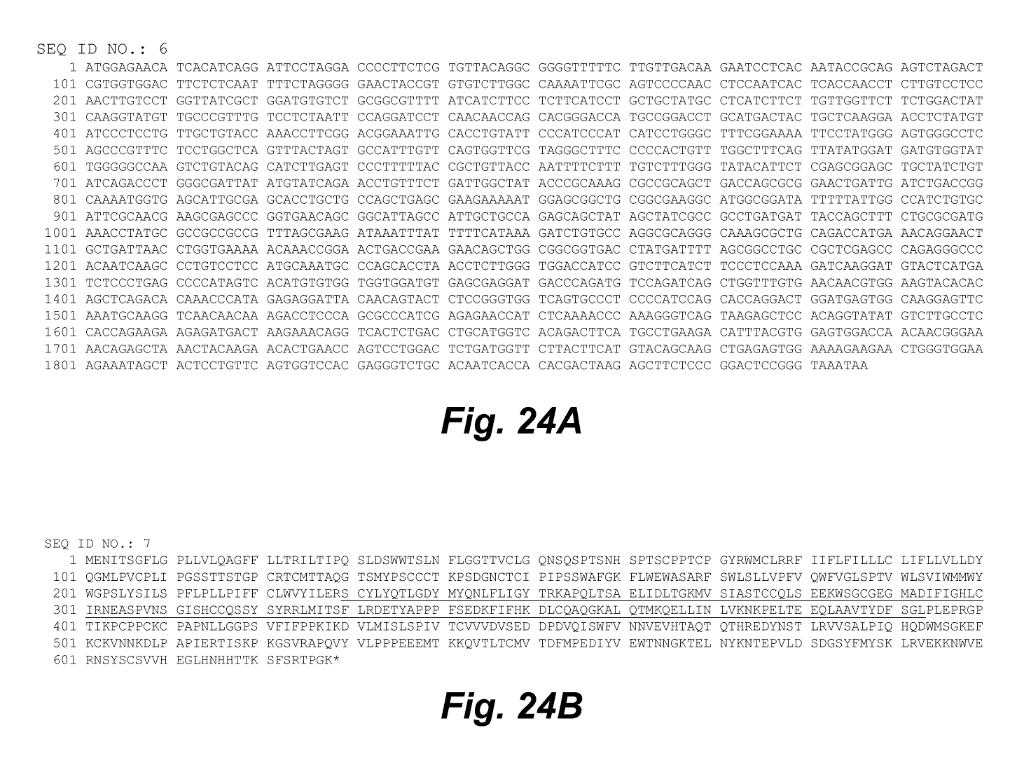

The amino acid sequence (SEQ ID No.: 2) of a HBsAg-TRP-1-Fc engineered fusion protein and the encoding nucleotide sequence (SEQ ID No.: 3) are illustrated in FIGS. 18 and 19, respectively. The increases in CD4 and CD8 immune responses are shown in FIG. 20.

1. A recombinant fusion protein for potentiating an immune system, the fusion protein comprising a self-assembling virus-like particle-forming polypeptide; an Fcγ receptor (FcγR)-binding ligand polypeptide, and a target antigen polypeptide, thereby forming a tripartite fusion polypeptide.

2. The recombinant fusion protein of claim 1, wherein the target antigen polypeptide is positioned between the self-assembling virus-like particle-forming polypeptide and the Fcγ receptor (FcγR)-binding ligand polypeptide.

3. The recombinant fusion protein of claim 1, wherein the self-assembling virus-like particle-forming polypeptide is selected from the group consisting of: an HPV L1 protein, an HPV L2 protein, an influenza Hemagglutinin A, an influenza neuraminidase, an influenza M1 protein, a lentivirus protein, any self-assembling fragment thereof, or any combination thereof.

4. The recombinant fusion protein of claim 1, wherein the target antigen polypeptide is a hepatitis B surface antigen (HBsAg) polypeptide, or fragment thereof.

5. The recombinant fusion polypeptide of claim 1, wherein the immunoglobulin Fcγ receptor-binding ligand domain comprises an immunoglobulin Fc domain.

6. The recombinant fusion polypeptide of claim 5, wherein immunoglobulin Fcγ receptor-binding ligand domain is isolated from an immunoglobulin G2a (IgG2a) or an immunoglobulin G1 (IgG1).

7. A nucleic acid encoding a recombinant fusion polypeptide according to claim 1.

8. The nucleic acid of claim 7, wherein the nucleic acid is inserted into an expression vector, and wherein the nucleic acid is operably linked to an expression control region for expression the nucleic acid in a recipient cell.

9. The nucleic acid of claim 7, wherein the nucleic acid is within a host cell selected from a mammalian cell, an insect cell, a yeast cell, and a prokaryotic cell.

10. An immunopotentiating virus-like particle (VLP) comprising a recombinant fusion protein according to claim 1.

11. A method of generating a plurality of virus-like particles (VLP) comprising a recombinant fusion protein, the method comprising:
(a) providing a nucleic acid encoding a recombinant fusion protein according to claim 1, wherein the nucleic acid is inserted into an expression vector, and wherein the nucleic acid is operably linked to an expression control region for expression the nucleic acid in a recipient cell;
(b) delivering the nucleic acid to a host cell suitable for expression of the nucleic acid from the expression vector, wherein the host cell is selected from a mammalian cell, an insect cell, a yeast cell, and a prokaryotic cell;
(c) expressing the nucleic acid in the host cell to provide said recombinant fusion protein; and
(d) allowing the recombinant fusion protein to self-assemble to form a plurality of VLPs.

12. The method of claim 11, further comprising the step of combining the plurality of VLPs with a physiologically acceptable carrier.

13. The method of claim 11, further comprising the step of combining the plurality of VLPs with an adjuvant.

14. An immunogenic composition comprising a plurality of immunopotentiating VLPs, wherein the immunopotentiating VLPs comprise a recombinant fusion protein according to claim 1, wherein said immunogenic composition is devoid of an adjuvant.

15. An immunogenic composition comprising a plurality of immunopotentiating VLPs, wherein the immunopotentiating VLPs comprise a recombinant fusion protein according to claim 1, wherein said immunogenic composition further comprises at least one adjuvant.

16. A method of inducing an immune response in an animal or human subject, comprising administering to the animal or human subject an immunogenic composition comprising a plurality of immunopotentiating VLPs comprising a recombinant fusion protein according to claim 1, whereby the VLPs are ingested by an antigen-presenting cell, thereby activating CD4 and CD8 T cells by MHC I and II.

17. The method of claim 16, wherein the activated CD4 and CD8 T cells invade a tumor, thereby reducing the extent of the tumor in the recipient animal or human subject.


SEQUENCE LISTING (9 sequences)

SEQ ID NO: 1 - protein, 459 aa, Artificial sequence
  HBs-Fc fusion antigen
MENITSGFLGPLLVLQAGFFLLTRILTIPQSLDSWWTSLNFLGGTTVCLGQNSQSPTSNHSPTSCPPTCPGYRWMCLRRFIIFLFILLLCLIFLLVLLDYQGMLPVCPLIPGSSTTSTGPCRTCMTTAQGTSMYPSCCCTKPSDGNCTCIPIPSSWAFGKFLWEWASARFSWLSLLVPFVQWFVGLSPTVWLSVIWMMWYWGPSLYSILSPFLPLLPIFFCLWVYIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 2 - protein, 538 aa, Artificial sequence
  HBsAg-TRP-1-Fc fusion antigen
MENITSGFLGPLLVLQAGFFLLTRILTIPQSLDSWWTSLNFLGGTTVCLGQNSQSPTSNHSPTSCPPTCPGYRWMCLRRFIIFLFILLLCLIFLLVLLDYQGMLPVCPLIPGSSTTSTGPCRTCMTTAQGTSMYPSCCCTKPSDGNCTCIPIPSSWAFGKFLWEWASARFSWLSLLVPFVQWFVGLSPTVWLSVIWMMWYWGPSLYSILSPFLPLLPIFFCLWVYILERGPGGPGSGHNCGTMRPGWRGAACNQKILTVRGPGTAPDNLGYMYEVQWPGQEFTVSEIITIAVVNALLGPGGPGPLEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 3 - DNA, 1617 bp, Artificial sequence
  HBsAg-TRP-1-Fc fusion antigen-encoding sequence
atggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacattctcgagcggggtccaggtggtccaggttcaggacacaactgtgggactatgcgtcctgggtggagaggagctgcatgcaaccagaaaattctcacagtcaggggtccaggtactgctccagacaatctgggatatatgtatgaagttcaatggccaggtcaggagtttactgtatctgaaatcattaccattgctgtagtgaacgcgttgttaggtccaggtggtccaggtccgctcgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaataa

SEQ ID NO: 4 - DNA, 1821 bp, Artificial sequence
  HBsAg-mAFP-Fc (S-A142-Fc) tripartite fusion antigen-encoding sequence
atggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacattctcgagcgggcggtgtttaacaaccgctttattctggaagtgagccgccgcaacccgtttatgtatgcgtatgccattctgagcctggcggcgcagtataacaaagtggtgctggcgtgctgcaaagcggataacaaagaagaatgctttcagaccaaacgcgcgagcattgcgaaagaactgcgcgaaggcagcatgctgaacgaacatgtgatgagcgtgattcgcaaatttggcagccgcaacctgcaggcgaccaccattattaaactgagccagaaactgaccgaagcgcagtttaccgaaattcagaaactggcgctggatgtggcgcatattcatgaagaatgctgccagggcaacaacctggaatgcctgcaggatggcgaaaaagtgatgacctatatttgcagccagcagaacattctgagcctgccgctcgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaataa

SEQ ID NO: 5 - protein, 606 aa, Artificial sequence
  HBsAg-mAFP-Fc (S-A142-Fc) tripartite fusion antigen
MENITSGFLGPLLVLQAGFFLLTRILTIPQSLDSWWTSLNFLGGTTVCLGQNSQSPTSNHSPTSCPPTCPGYRWMCLRRFIIFLFILLLCLIFLLVLLDYQGMLPVCPLIPGSSTTSTGPCRTCMTTAQGTSMYPSCCCTKPSDGNCTCIPIPSSWAFGKFLWEWASARFSWLSLLVPFVQWFVGLSPTVWLSVIWMMWYWGPSLYSILSPFLPLLPIFFCLWVYILERAVFNNRFILEVSRRNPFMYAYAILSLAAQYNKVVLACCKADNKEECFQTKRASIAKELREGSMLNEHVMSVIRKFGSRNLQATTIIKLSQKLTEAQFTEIQKLALDVAHIHEECCQGNNLECLQDGEKVMTYICSQQNILSLPLEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 6 - DNA, 1887 bp, Artificial sequence
  HBsAg-mAFP-Fc (S-A164-Fc) tripartite fusion antigen-encoding sequence
atggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacattctcgagcggagctgctatctgtatcagaccctgggcgattatatgtatcagaacctgtttctgattggctatacccgcaaagcgccgcagctgaccagcgcggaactgattgatctgaccggcaaaatggtgagcattgcgagcacctgctgccagctgagcgaagaaaaatggagcggctgcggcgaaggcatggcggatatttttattggccatctgtgcattcgcaacgaagcgagcccggtgaacagcggcattagccattgctgccagagcagctatagctatcgccgcctgatgattaccagctttctgcgcgatgaaacctatgcgccgccgccgtttagcgaagataaatttatttttcataaagatctgtgccaggcgcagggcaaagcgctgcagaccatgaaacaggaactgctgattaacctggtgaaaaacaaaccggaactgaccgaagaacagctggcggcggtgacctatgattttagcggcctgccgctcgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaataa

SEQ ID NO: 7 - protein, 628 aa, Artificial sequence
  HBsAg-mAFP-Fc (S-A164-Fc) tripartite fusion antigen
MENITSGFLGPLLVLQAGFFLLTRILTIPQSLDSWWTSLNFLGGTTVCLGQNSQSPTSNHSPTSCPPTCPGYRWMCLRRFIIFLFILLLCLIFLLVLLDYQGMLPVCPLIPGSSTTSTGPCRTCMTTAQGTSMYPSCCCTKPSDGNCTCIPIPSSWAFGKFLWEWASARFSWLSLLVPFVQWFVGLSPTVWLSVIWMMWYWGPSLYSILSPFLPLLPIFFCLWVYILERSCYLYQTLGDYMYQNLFLIGYTRKAPQLTSAELIDLTGKMVSIASTCCQLSEEKWSGCGEGMADIFIGHLCIRNEASPVNSGISHCCQSSYSYRRLMITSFLRDETYAPPPFSEDKFIFHKDLCQAQGKALQTMKQELLINLVKNKPELTEEQLAAVTYDFSGLPLEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 8 - DNA, 20 bp, Artificial sequence
  IL-2 forward primer
cccttgctaatcactcctca

SEQ ID NO: 9 - DNA, 20 bp, Artificial sequence
  IL-2 reverse primer
gagctcctgtaggtccatca